(19) United States
(12) Patent Application Publication
(10) Pub. No.: US 2009/0110679 A1
(43) Pub. Date: Apr. 30, 2009

(54) Methods and compositions for pulmonary administration of a TNFa inhibitor

(76) Inventors: Luk-Chiu Li, Lake Forest, IL (US); Yi Shi, Libertyville, IL (US); Thomas L. Reiland, Scottsdale, AZ (US); Masahiro Sakagami, Richmond, VA (US); Katherine Nicholson, Chesterfield, VA (US); Peter R. Byron, Richmond, VA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/217,972

(22) Filed: Jul. 10, 2008

Related U.S. Application Data
(60) Provisional application No. 60/959,426, filed on Jul. 13, 2007.

(51) Int. Cl. A61K 39/395; A61K 39/00; A61M 15/00

(52) U.S. Cl. 424/133.1; 424/130.1; 424/178.1; 128/203.15; 128/200.23

(57) ABSTRACT

The invention describes methods of pulmonary delivery of a TNFα inhibitor to a subject having a disorder in which TNFα is detrimental, such that the disorder is treated. Also included is a method of achieving systemic circulation of a TNFα inhibitor in a subject comprising administering the TNFα inhibitor to the central lung region or the peripheral lung region of the subject via inhalation, such that systemic circulation of the TNFα inhibitor is achieved.

### CROSS REFERENCE TO RELATED APPLICATION

This application claims priority to U.S. Provisional Application Ser. No. 60/959,426 filed on Jul. 13, 2007, which is incorporated herein in its entirety.

### BACKGROUND OF THE INVENTION

Due to the large molecular size of many therapeutic biologics, e.g., 150 kd antibody, the therapeutically effective route of administration is generally limited to invasive injections, which often can be painful, especially in view of the fact that often the patients who rely on therapeutic biologics are treating chronic diseases. Thus, there remains a need for less painful but effective ways of delivering therapeutic biologics to patients.

### SUMMARY OF THE INVENTION

The invention provides an improved method for systemically delivering a TNFα inhibitor to a subject, wherein the delivery method decreases pain often associated with injections. The invention also provides a method for delivering a TNFα inhibitor locally to the lung of a subject for treatment of a pulmonary disorder.

The invention includes a method of treating a subject having a disorder in which TNFα activity is detrimental comprising pulmonary delivery of a TNFα inhibitor to the subject, such that the disorder in which TNFα is detrimental is treated. The invention also includes a method of achieving systemic circulation of a TNFα inhibitor in a subject comprising administering the TNFα inhibitor to the central and peripheral lung region of the subject via inhalation, such that systemic circulation of the TNFα inhibitor is achieved. The invention further provides a method of achieving systemic circulation of a TNFα inhibitor in a subject comprising administering the TNFα inhibitor to the peripheral lung region of the subject via inhalation, such that systemic circulation of the TNFα inhibitor is achieved.

The invention also includes a method of treating a pulmonary disorder in a subject comprising pulmonary delivery of a TNFα inhibitor to the subject, wherein the pulmonary administration comprises local delivery of the TNFα inhibitor to the lung(s) of the subject.

The TNFα inhibitor may be formulated in a composition suitable for inhalation, including, for example, an inhalable powder, a propellant-containing aerosol, and a propellant-free inhalable solution. In one embodiment, the inhalable powder is administered to the subject via a dry powder inhaler (DPI). In one embodiment, the propellant-containing aerosol is administered to the subject via a metered dose inhaler (MDI). In one embodiment, the propellant-free inhalable solution is administered to the subject via a nebulizer.

In one embodiment, the invention further comprises achieving certain pharmacokinetic parameters for pulmonary delivery of a TNFα inhibitor. For example, in one embodiment, the invention includes a method of achieving a Tₘₐₓ of less than or equal to about 4 days for the TNFα inhibitor. In another embodiment, the TNFα inhibitor is distributed to the central lung region of the subject such that a P/C ratio of about 0.3 is achieved. In still another embodiment, the TNFα inhibitor is distributed to the peripheral lung region of the subject such that a P/C ratio of about 1.3 is achieved.

In yet another embodiment, a maximum serum concentration (Cₘₐₓ) of at least about 2.3 mg/L of the TNFα inhibitor is achieved. In one embodiment, a Cₘₐₓ of at least about 4.2 mg/L of the TNFα inhibitor is achieved. In another embodiment, a Cₘₐₓ of at least about 5 mg/L of the TNFα inhibitor is achieved. In still another embodiment, at least one pharmacokinetic characteristic selected from the group consisting of a Tₘₐₓ of less than or equal to about 4 days, an absolute bioavailability (F %) of at least about 0.99%, and a Cₘₐₓ of at least about 2.3 mg/L, is achieved following administration of the TNFα inhibitor. In one embodiment, a Tₘₐₓ of about 2 to about 4 days is achieved following administration of the TNFα inhibitor. In one embodiment, a Cₘₐₓ of about 2.3 to about 5.9 mg/L is achieved following administration of the TNFα inhibitor.

The invention also includes pharmaceutical compositions suitable for delivering a TNFα inhibitor to the lung of a subject. The invention provides a pharmaceutical composition comprising a TNFα antibody and a pharmaceutically acceptable carrier, wherein the pharmaceutical composition is suitable for inhalation by a subject and is selected from the group consisting of an inhalable powder or a dry powder composition, a propellant-containing aerosol, and a propellant-free inhalable solution or suspension. In one embodiment, the pharmaceutically acceptable carrier comprises a lactose powder or a glucose powder.

The invention further provides devices or containers comprising a TNFα inhibitor which are suitable for pulmonary administration of the TNFα inhibitor. The invention provides a dry powder inhaler (DPI) device for pulmonary administration of a TNFα inhibitor to a subject, the DPI device comprising a reservoir comprising an inhalable powder or dry powder composition comprising the TNFα inhibitor, and a means for introducing the inhalable powder or dry powder composition into the subject via inhalation. In one embodiment, the DPI device is either a single dose or a multidose inhaler. In another embodiment, the DPI device is either pre-metered or device-metered.

The invention also provides a metered dose inhaler (MDI) device for pulmonary administration of a TNFα inhibitor to a subject, the MDI device comprising a pressurized canister comprising an aerosol comprising the TNFα inhibitor and a propellant, and a means for introducing the aerosol into the subject via inhalation.

The invention further provides a container for use with a nebulizer device for pulmonary administration of a TNFα inhibitor to a subject, the container comprising a propellant-free inhalable solution or suspension comprising the TNFα inhibitor.

The invention also includes modified TNFα antibody, or antigen-binding portion thereof, which have decreased binding to phagocytic receptors expressed on alveolar macrophage. Also included in the invention is a modified TNFα antibody, or antigen-binding portion thereof, which has enhanced binding to a neonatal Fc receptor (FcRN). In one embodiment, the modified TNFα antibody is conjugated to a compound which increases transport of the TNFα antibody from the lung epithelium of a subject to the bloodstream of the subject. In another embodiment, the modified TNFα antibody comprises mutations and/or deletions within the Fc domain which increase the binding affinity of the TNFα antibody to FcRn, including, for example, at least one mutation within the Fc domain at an amino acid position selected from the group consisting of 238, 256, 307, 311, 312, 380, and 382.

In one embodiment, the subject is a human.

In one embodiment, the subject has a disorder in which TNFα activity is detrimental, including, for example, an autoimmune disorder, a spondyloarthropathy, an intestinal disorder, a skin disorder, and a pulmonary disorder.

In one embodiment, the autoimmune disorder is rheumatoid arthritis or juvenile rheumatoid arthritis.

In one embodiment, the spondyloarthropathy is ankylosing spondylitis or psoriatic arthritis.

In one embodiment, the intestinal disorder is Crohn's disease.

In one embodiment, skin disorder is psoriasis.

In one embodiment, pulmonary disorder is chronic obstructive pulmonary disease or asthma.

In one embodiment, the TNFα inhibitor is a TNFα antibody, or antigen-binding portion thereof, or a fusion protein.

In one embodiment, the fusion protein is etanercept.

In one embodiment, the TNFα antibody, or antigen-binding portion thereof, is selected from the group consisting of infliximab, golimumab, and adalimumab.

In one embodiment, the TNFα antibody, or antigen-binding portion thereof, is an antibody selected from the group consisting of a humanized antibody, a chimeric antibody, a human antibody, and a multivalent antibody.

In one embodiment, the human TNFα antibody, or antigen-binding portion thereof, dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

In one embodiment, the human TNFα antibody, or antigen-binding portion thereof, has the following characteristics: dissociates from human TNFα with a K_{off} rate constant of 1×10⁻³ s⁻¹ or less, as determined by surface plasmon resonance; has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9; and has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In one embodiment, the human TNFα antibody, or antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

In one embodiment, the human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

In one embodiment, the methods and compositions of the invention comprise at least about 40 mg of the TNFα antibody, or antigen-binding portion thereof. In another embodiment, the methods and compositions of the invention comprise about 40-160 mg of the TNFα antibody, or antigen-binding portion thereof.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 shows regional tissue dissection of monkey lung into the tracheobronchi (TB) and the central (C) and peripheral (P) lung lobar regions to determine lung-regional distributions following 2 different (shallow and deep) modes of inhalation.

FIG. 2 graphically depicts serum adalimumab concentration vs. time profiles following two modes of inhalation at a nominal 10 mg/kg of the lung-deposited dose in 4 monkeys. Each profile represents each individual animal assigned to receive adalimumab aerosols into the lung via shallow (closed symbols) and deep (open symbols) inspiratory maneuvers.

FIG. 3 graphically depicts serum adalimumab concentration vs. time profiles following intravenous injection at a dose of 10 mg/kg in 2 monkeys. Each profile represents each individual animal.

FIG. 4 shows the lung-regional distribution of FD-150S following (a) shallow and (b) deep inspiratory maneuvers alongside manipulations in intubation depth and aerosol size at a nominal dose of 2.5 mg/kg in monkeys. The data represent mean % of deposition from 3 animals in the tracehobronchial (TB), central (C) and peripheral (P) regions of the lung.

### DETAILED DESCRIPTION OF THE INVENTION

### I. Definitions

The term “pulmonary administration” or “pulmonary delivery” refers to administration of a TNFα inhibitor through the lungs of a subject by inhalation.

As used herein, the term “inhalation” refers to intake of air to the lung. In specific examples, intake can occur by self-administration of a formulation comprising a TNFα inhibitor while inhaling, or by administration via a respirator, e.g., to a patient on a respirator. The term “inhalation” used with respect to a formulation is synonymous with “pulmonary administration.”

The term “central lung region” or “central airway,” as used herein, refers to a conducting or transitional airway, distal to the larynx, which has little to no role in gas exchange. In humans central airways include the trachea, main bronchi, lobar bronchi, segmental bronchi, small bronchi, bronchioles, terminal bronchioles, and respiratory bronchioles. The central airways thus account for the first 16-19 generations of airway branching in the lung, where the trachea is generation zero (0) and the alveolar sac is generation 23 (Wiebel (1963) Morphometry of the Human Lung, Berlin:Springer-Verlag, pp. 1-151). The central airways are responsible for the bulk movement of air, as opposed to the periphery of the lung, which is primarily responsible for gas exchange between air and blood. In one embodiment, a TNFα inhibitor is targeted to the central lung region through shallow inhalation.

The “peripheral lung region” or “peripheral airway,” as used herein, refers to airways of the lung distal to the central airways.

The term “Cₘₐₓ” refers to the maximum or peak serum or plasma concentration of an agent observed in a subject after its administration.

The term “Tₘₐₓ” refers to the time at which Cₘₐₓ occurred.

The term “bioavailability” or “F %” refers to a fraction or percent of a dose which is absorbed and enters the systemic circulation after administration of a given dosage form. The dose of the agent may be administered through any route other than the intravenous route, and, preferably, via pulmonary delivery.

As used herein, the term “P/C ratio” or “P/C” refers to a measure of relative distribution of deposition of an agent, e.g., TNFα inhibitor, to the periphery of the lung in comparison to the central lung region.

As used herein, the term “aerosol” refers to solid and/or liquid suspension in the air. In particular, aerosol refers to the particalization of a formulation of the invention and its suspension in the air. According to the present invention, an aerosol formulation is a formulation comprising a complement inhibitory protein that is suitable for aerosolization, i.e., particlization and suspension in the air, for inhalation or pulmonary administration.

The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFαinhibitor is a recombinant TNF binding protein (r-TBP-I) (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH or VL domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions which may be used in the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 0.90/07861, and Winter, U.S. Pat. No. 5,225,539.

An “isolated antibody”, as used herein, is intended to refer to an antibody that is substantially free of other antibodies having different antigenic specificities (e.g., an isolated antibody that specifically binds hTNFα is substantially free of antibodies that specifically_bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit 50% of the maximum biological endpoint of interest, e.g., neutralization of cytotoxicity activity.

The term “dose,” as used herein, refers to an amount of TNFα inhibitor which is administered to a subject.

The term “dosing”, as used herein, refers to the administration of a substance (e.g., an anti-TNFα antibody) to achieve a therapeutic objective (e.g., treatment of disease in which TNFα activity is detrimental).

A “dosing regimen” describes a treatment schedule for a TNFα inhibitor, e.g., a treatment schedule over a prolonged period of time and/or throughout the course of treatment. In one embodiment, the dosing regimen comprises administering a first dose of a TNFα inhibitor via pulmonary administration at week 0 followed by a second dose of a TNFα inhibitor via pulmonary administration on a biweekly dosing regimen.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. Preferably, the substance is administered every 9-19 days, more preferably, every 11-17 days, even more preferably, every 13-15 days, and most preferably, every 14 days. In one embodiment, the biweekly dosing regimen is initiated in a subject at week 0 of treatment. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “multiple-variable dose” includes different doses of a TNFα inhibitor which are administered to a subject for therapeutic treatment. “Multiple-variable dose regimen” or “multiple-variable dose therapy” describes a treatment schedule which is based on administering different amounts of a TNFα inhibitor at various time points throughout the course of treatment. Multiple-variable dose regimens are described in PCT application no. PCT/US05/12007 and US 20060009385, which is incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions, wherein one or both of the agents is delivered via pulmonary administration.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

The term “treatment,” as used within the context of the present invention, is meant to include therapeutic treatment, as well as prophylactic or suppressive measures, for the treatment of a disorder in which TNFα activity is detrimental. For example, the term treatment may include pulmonary administration of a TNFα inhibitor prior to or following the onset of a disorder in which TNFα activity is detrimental, thereby preventing or removing signs of the disease or disorder. As another example, administration of a TNFα inhibitor after clinical manifestation of a disorder in which TNFα activity is detrimental to combat the symptoms and/or complications and disorders associated with the disorder in which TNFα activity is detrimental comprises “treatment” of the disease. Further, pulmonary administration of the agent after onset and after clinical symptoms and/or complications have developed where administration affects clinical parameters of the disease or disorder and perhaps amelioration of the disease, comprises “treatment” of the a disorder in which TNFα activity is detrimental.

Those “in need of treatment” include mammals, such as humans, already having a disorder in which TNFα activity is detrimental, including those in which the disease or disorder is to be prevented.

Various aspects of the invention are described in further detail herein.

### II. Methods and Compositions for Pulmonary Administration

Pulmonary administration of a TNFα inhibitor, e.g., a TNFα antibody, provides an advantageous alternative to more traditional means of drug delivery, e.g., subcutaneous and intravenous. By inhaling a TNFα inhibitor for treatment of a disorder, a subject is able to avoid the pain associated with a needle injection, but still achieve systemic circulation of the TNFα inhibitor resulting in a therapeutic effect.

Thus, the invention is directed to methods and compositions for administration of a TNFα inhibitor, e.g., a TNFα antibody, to a subject via pulmonary administration. The invention is also directed to a method of treating a subject having a disorder in which TNFα activity is detrimental comprising pulmonary delivery of a TNFα inhibitor to the subject, such that the disorder in which TNFα is detrimental is treated.

The invention also provides certain pharmacokinetic parameters which are result in successful pulmonary delivery of a TNFα inhibitor, e.g., a TNFα antibody, such that the TNFα inhibitor reaches therapeutically desired serum levels. In one embodiment, the TNFα inhibitor, e.g., a TNFα antibody, is delivered via inhalation to a subject, such that a Tₘₐₓ of less than or equal to about 4 days is achieved. In another embodiment, inhalation of the TNFα inhibitor results in a maximum serum concentration (Cₘₐₓ) of at least about 2.3 mg/l of the TNFα inhibitor. In one embodiment, a Cₘₐₓ of at least about 2.3 mg/l, 2.4 mg/l, 2.5 mg/l, 2.6 mg/l, 2.7 mg/l, 2.8 mg/l, 2.9 mg/l, 3.0 mg/l, 3.1 mg/l, 3.2 mg/l, 3.3 mg/l, 3.4 mg/l, 3.5 mg/l, 3.6 mg/l, 3.7 mg/l, 3.8 mg/l, 3.9 mg/l, 4.0 mg/l, 4.1 mg/l, 4.2 mg/l, 4.3 mg/l, 4.4 mg/l, 4.5 mg/l, 4.6 mg/l, 4.7 mg/l, 4.8 mg/l, 4.9 mg/l, and 5.0 mg/l, 5.1 mg/l, 5.2 mg/l, 5.3 mg/l, 5.4 mg/l, 5.5 mg/l, 5.6 mg/l, 5.7 mg/l, 5.8 mg/l, 5.9 mg/l, and 6.0 mg/l is achieved. Other pharmacokinetic characteristics included in the invention which result in therapeutic levels of the TNFα inhibitor, e.g., a human the TNFα antibody, in the serum of a subject who has received the TNFα inhibitor via pulmonary means include a Tₘₐₓ of less than or equal to about 4 days, a Tₘₐₓ of about 2 to about 4 days, an absolute bioavailability (F %) of at least about 0.99%, a Cₘₐₓ of about 2.3 to about 5.9 mg/L, and a Cₘₐₓ of at least about 2.3 mg/L.

The invention also includes pulmonary delivery of a TNFα inhibitor to a subject such that systemic circulation of the TNFα inhibitor is achieved, wherein the TNFα inhibitor is delivered either to the central lung region or to the peripheral lung region. In accordance with the methods of the invention, systemic circulation via pulmonary administration of a TNFα inhibitor, e.g., a TNFα antibody, may be achieved either through the central lung region, the peripheral lung region, or both lung regions. Thus, in one embodiment, the invention is directed to a method of achieving systemic circulation of a TNFα inhibitor in a subject comprising administering the TNFα inhibitor to the central lung region of the subject via inhalation, such that systemic circulation of the TNFα inhibitor is achieved. In another embodiment, the invention is directed to a method of achieving systemic circulation of a TNFα inhibitor in a subject comprising administering the TNFα inhibitor to the peripheral lung region of the subject via inhalation, such that systemic circulation of the TNFα inhibitor is achieved.

In one embodiment, inhalation of the TNFα inhibitor by a subject is targeted to the central lung region of the subject in order to achieve systemic circulation of the TNFα inhibitor. Studies have suggested that carrier-mediated systemic absorption of Fc fusion proteins from the lung airways may be possible (Bitonti et al. (2004) PNAS 101:9763). It is believed that this absorption is transctytosis mediated via its specific binding transporter, the neonatal constant regions fragment (Fc) receptor (FcRn), while predominating from the bronchial airways where FcRn localization seems to be more abundant (Bitonti et al. (2004)). WO 04/004798 (2004) describes aerosol delivery to the central lung region of Fc fusion proteins, including EPO-Fc. The invention describes means for achieving successful central airway delivery of a TNFα inhibitor, i.e., a TNFα antibody, thus demonstrating that a TNFα antibody may be administered to a subject by inhalation.

In one embodiment, inhalation of the TNFα inhibitor by a subject is targeted to the peripheral region of the subject in order to achieve systemic circulation of the TNFα inhibitor. The peripheral lung region has been shown to be advantageous for absorption of agents delivered via inhalation, as the peripheral lung region has the greatest amount of surface area available for absorption (see Yu et al. (1997) Crit. Rev Therapeutic Drug Carrier Systems 14:395).

The P/C ratio represents the penetration index as a measure of effective administration of an agent to the peripheral lung. In one embodiment, the invention provides a method of achieving systemic circulation of a TNFα inhibitor, wherein the TNFα inhibitor is distributed to the central lung region of the subject such that a P/C ratio of about 0.3 is achieved. In one embodiment, the invention provides a method of achieving systemic circulation of a TNFα inhibitor, wherein the TNFα inhibitor is distributed to the peripheral lung region of the subject such that a P/C ratio of about 1.3 is achieved.

Pulmonary administration may be accomplished by suitable means known to those in the art. Pulmonary administration of a TNFα inhibitor requires dispensing of the biologically active substance from a delivery device into the oral cavity of a subject during inhalation. For purposes of the present invention, compositions comprising a TNFα inhibitor are administered via inhalation of an aerosol or other suitable preparation that is obtained from an aqueous or nonaqueous solution or suspension form, or a solid or dry powder form of the pharmaceutical composition, depending upon the delivery device used. Such delivery devices are well known in the art and include, but are not limited to, nebulizers, metered-dose inhalers, and dry powder inhalers, or any other appropriate delivery mechanisms that allow for dispensing of a pharmaceutical composition as an aqueous or nonaqueous solution or suspension or as a solid or dry powder form.

Methods for delivering a TNFα inhibitor, including a TNFα antibody, or antigen-binding portion thereof, to a subject via pulmonary administration, including directed delivery to the central and/or peripheral lung region(s), include, but are not limited to, a dry powder inhaler (DPI), a metered dose inhaler (MDI) device, and a nebulizer.

### Dry Powder Inhaler (DPI) Devices

In one embodiment, the TNFα inhibitor, including a TNFα antibody, or antigen-binding portion thereof, is delivered to a subject through a dry powder inhaler (DPI). A DPI is used to deliver an agent, such as a TNFα inhibitor, in a solid or dry powder form using a subject's inspiration to deliver the dry powder to the lungs, instead of a mist. A DPI is used to breathe in (inhale) a TNFα inhibitor so that it goes directly into the subject's lungs. A DPI is a propellant-free device, wherein the agent for delivery is blended with suitable carriers known in the art. The unit dose of agent used in a DPI device is often a dry powder blister disc of hard capsule. A DPI produces dispersible and stable dry powder formulations which are inhaled, including spray drying, spray-freeze drying, and micronized milling formulations. DPI devices have been used to deliver macromolecular agents, including insulin, interferon (IFN), and growth hormone (GH).

Examples of DPI devices include, but are not limited to, the following:

The AIR® inhaler (Alkermes) which includes a small, breath-activated system that delivers porous powder from a capsule (see WO 99/66903 and WO 00/10541). The porous particles have an aerodynamic diameter of 1-5 μm and are prepared by spray drying. The AIR™ inhaler has been used to deliver albuterol, epinephrine, insulin, and hGH.

The TurboHaler® (AstraZeneca) is also a DPI which may be used in the methods of the invention and is described in EP patent 0799067, incorporated by reference herein. This DPI device is an inspiratory flow-driven, multidose dry-powder inhaler with a multi-dose reservoir that provides up to 200 doses of the drug formulation and dose ranges from a few micrograms to 0.5 mg. Examples the TurboHaler™ include Pulmicort® (also Pulmicort® TurbuHaler®) which delivers budesonide an anti-inflammatory glucocorticosteroid indicated for the once or twice-daily maintenance treatment of asthma, Oxis® (formoterol) which is a fast-acting beta2-agonist with a long duration for once- or twice-daily maintenance therapy of asthma, and Symbicort® (budesonide/formoterol), which contains the corticosteroid budesonide and rapid- and long-acting bronchodilator formoterol in a single inhaler.

Eclipse™ (Aventis) represents a breath actuated re-usable capsule device capable of delivering up to 20 mg of formulation. The powder is sucked from the capsule into a vortex chamber where a rotating ball aids in powder disaggregation as the subject inhales (see U.S. Pat. No. 6,230,707 and WO9503846).

Another DPI device which may be used in the methods and compositions of the invention includes the Ultrahaler® (Aventis) which combines accurate dose metering and good dispersion in a device providing one month's therapy in an easy to use, discrete, pocket sized device with a numerical dose counter, dose taken indicator and a lock-out mechanism. The device is capable of delivery up to 20 mg of formulation. The Ultrahaler® described in U.S. Pat. No. 5,678,538 and WO2004026380.

Another DPI device, which may be used in the methods and compositions of the invention includes the Bang Olufsen breath actuated inhaler, which is a breath actuated inhaler using blister strips with up to sixty doses. The dose is made available only during the inhalation by a novel trigger mechanism. The device is equipped with a dose counter and is most likely disposed after all doses have been used. (see EP 1522325).

An active DPI (also usable as an MDI—described below) described in WO 94/19042 (Bespak) includes a device which is hand held, self contained unit that employs multiple, carbon fiber brush, setaceous electrodes to disperse powders and aerosols into fine/particles/mists. As the patient inhales, 1 to 10 kvolts is passed through the electrodes to disperse the powder/aerosol. A breath sensor comprised of a piezoelectric membrane that flexes in response to a change of air pressure in the passageway and thereby generate a signal representative of inhalation being sensed is employed to initiate the electric discharge.

The HandiHaler® (Boehringer Ingelheim GmbH) is a single dose DPI device, which can deliver up to 30 mg of formulated drug in capsules (see WO2004024156). An example of this device is Spiriva® (tiotropium bromide) which delivers 3.6 mcg of the 18 mcg dose to the lung.

The PADD DPI (Britannia Pharmaceuticals) is a pressurized aerosol dry powder delivery device capable of delivering up to 100 mg formulation. The system utilizes a novel formulation comprised of surface active phospholipids, dipalmitoyl phosphatidyl choline (DPPC) and phosphatidyl glycerol (PG), prepared in the form of a fine powder. The PADD device offers the highest payload possible with a propellant powered device. (see U.S. Pat. No. 6,482,391).

Another DPI device, which may be used in the methods and compositions of the invention includes the Pulvinal® inhaler (Chiesi) which is a breath-actuated multi-dose (100 doses) dry powder inhaler (see U.S. Pat. No. 5,351,683). The dry powder of the drug is stored in a reservoir which transparent and clearly marked to indicate when the 100th dose has been delivered. The Pulvinal inhaler has been used to deliver respiratory drugs such as salbutamol (Butovent® Pulvinal®), beclomethasone (Clenil® Pulvinal®) as well as budesonide and formoterol.

Yet another DPI device which may be used in the methods and compositions of the invention includes NEXT DPI™, which is a device which features multidose capabilities, moisture protection, and dose counting. The device can be used regardless of orientation (upside down), dose only when proper aspiratory flow is reached (see EP1196146, U.S. Pat. No. 6,528,096, WO0178693, WO0053158).

The DirectHaler™ (Direct-Haler A/S) may also be used in the methods and compositions of the invention (see U.S. Pat. No. 5,797,392). This device is a single dose, pre-metered, pre-filled, disposable DPI device made from polypropylene. The device is 72 mm in length, transparent, single dose, DPI device that resembles a straw, and has been used to deliver formulations of budesonide and formoterol.

The Accuhaler/Diskus™ (GlaxoSmithKline) is a disposable small DPI device that holds up to 60 doses contained in double foil blister strips to provide moisture protection (see GB2242134). It has been used to deliver flutacasone propionate/salmeterol xinafoate, flutacasone propionate, salmeterol xinafoate, and salbutamol.

In addition, the methods may include the FlowCaps® (Hovione) which is a capsule-based, re-fillable, re-usable passive dry-powder inhaler that holds up to 14 capsules. The FlowCaps® is pen-shaped, and measures about 11 cm in length, 2 cm in diameter. The inhaler itself is moisture-proof (see U.S. Pat. No. 5,673,686).

In one embodiment, the DPI device used in the invention is a Clickhaler® (Innovata PLC), which is a large reservoir breath-activated multidose device (see U.S. Pat. No. 5,437,270). It is used to treat asthma and COPD with a variety of drugs, including salbutamol (Asmasal®), beclomethasone (Asmabec®), and procaterol hydrochloride (Meptin®) as well as budesonide and formoterol. Another DPI device comprising a reservoir which may be used in the invention includes the Duohaler® (Innovata PLC) which is a fixed combination therapy, multi-dose DPI (see WO0139823). It has two separate reservoirs which feed two separate formulations into separate metering chambers from which the drugs are delivered to the patient in the same breath; this approach overcomes co-formulation issues. Duohaler® is therefore ideally suited for the delivery of fixed combination therapy for asthma and COPD.

In one embodiment, the DPI device used in the invention is an S2 unit dose (Innovata PLC), which is a re-useable or disposable single-dose DPI for the delivery of a wide range of therapeutics in high concentrations. Its dispersion mechanism means that minimal patient effort is required to ensure excellent drug delivery to the patients' lungs, a feature of particular benefit to systemic drug delivery. S2 is easy to use and has a passive engine so no battery or power source is required (see AU3320101).

Yet another DPI device which may be used in the methods and compositions of the invention includes Taifun® DPI (LAB International) which is a multiple-dose (up to 200) DPI device that is breath actuated and flow rate independent. The device consists of a unique moisture-balancing drug reservoir coupled with patented volumetric dose metering system for consistent dosing (see U.S. Pat. No. 6,132,394).

In one embodiment, the DPI device used in the invention is MedTone® (Mannkind Corp., see WO0107107) which comprises an intake section, a mixing section, and a mouthpiece. The mouthpiece is connected by a swivel joint to the mixing section. The intake chamber comprises a piston with a tapered piston rod and spring, and one or more bleed-through orifices to modulate the flow of air through the device. The mixing section holds a capsule with holes containing a dry powder medicament, and further opens and closes the capsule when the intake section is at a certain angle to the mouthpiece. The mixing section is a Venturi chamber to impart a cyclonic flow to air passing through the mixing chamber. The mouthpiece includes a tongue depressor, and a protrusion to contact the lips of the user to tell the user that the DPI is in the correct position. Technosphere® Insulin System, used for the treatment of diabetes, consists of a dry-powder Technosphere® formulation (see US2004096403) of insulin and MedTone® inhaler through which the powder is inhaled into the deep lung. The powder formulation of the drug to be delivered in microparticles has a size range between 0.5 and ten microns, preferably in the range of two to five microns, formed of a material releasing drug at a pH of greater than 6.4.)

Yet another DPI device which may be used in the methods and compositions of the invention includes Xcelovair™ (Meridica/Pfizer) and features 60 pre-metered, hermetically sealed doses in the range of 5-20 mg. The device provides moisture protection under accelerated conditions of 40° C./75% RH. The dispersion system maximizes the fine particle fraction delivery to achieve up to 50% fine particle mass.

Yet another DPI device which may be used in the methods and compositions of the invention includes MicroDose® DPI (Microdose Technologies) which is a small electronic DPI device that uses piezoelectric vibrator (ultrasonic frequencies) to deaggragate the drug powder (small or large molecules, neat chemical or mixtures of drug and lactose up to 3 mg drug) in an aluminum blister (single or multiple dose) (see U.S. Pat. No. 6,026,809). It has been used for pulmonary delivery of insulin.

In one embodiment, the DPI device used in the invention is Nektar Pulmonary Inhaler® (Nektar) which was designed to efficiently remove powders from the packaging, break up the particles and create an aerosol cloud suitable for deep lung delivery (see AU4090599, U.S. Pat. No. 5,740,794). It is designed to enable the aerosolized particles to be transported from the device to the deep lung during a patient's breath, reducing losses in the throat and upper airways. Compressed gas is used to aerosolize the powder. This DPI device is used in Exubera® inhalable insulin (Pfizer, Sanofi-Aventis, and Nektar), as well as to administer tobramycin, leuprolide, and single chain antibodies.

Also included in the invention is the Nektar Dry Powder Inhaler® (Nektar) which is palm-sized and easy to use, and provides convenient dosing from standard capsules and flow-rate-independent lung deposition when used in combination with Nektar Pulmonary Technology® (see US2003094173). Appropriate for either large or small molecules, the Nektar DPI is ideal for large payloads (2-50 mg). This disposable device is designed for short-term use. This device has been used to deliver tobramycin inhalation powder for ling infections in patients with Cystic Fibrosis and inhaled amphotericin B for treatment of fungal infection.

Also included in the invention is the Oriel™ DPI which is an active DPI that utilizes a piezoelectric membrane and nonlinear vibrations to aerosolize powder formulations (see WO0168169).

In addition, EasyHaler® (Orion Pharma) may be used in the methods and compositions of the invention. EasyHaler® is a multidose dry powder inhaler for lung and nasal delivery, and provides good performance for local lung delivery (see WO02102444), although moisture protection is not available for sensitive drugs. The EasyHaler® includes Beclomet EasyHaler®/Atomide EasyHaler® (beclomethasone dipropionate) and Buventol EasyHaler®/Salbu EasyHaler® (salbutamol).

Also included in the invention is the Jethaler® (Pulmotec) which utilizes the MAG (mechanical aerosol generation) technology for CFC-free dry-powder inhalation. The MAG technology is based on the principle of mechanical aerosol generation, in which the dosage for the inhalation is mechanically generated from a highly compressed solid. The JetHaler® has been used to deliver budesonide (Budesonid-ratiopharm®).

Yet another DPI device which may be used in the methods and compositions of the invention includes Accu-Breathe™ single dose DPI (Respirics), which is a single dose device that delivers dose when a predetermined inspiratory flow rate achieved (see WO03035137, U.S. Pat. No. 6,561,186). This DPI device is capable of delivering multiple formulations using novel dual capsule packaging system.

Also included in the invention is the Acu-Breather™ multidose DPI (Respirics) which uses an aclar/PVC moisture protected blister cartridge capable of holding 25-50 mg of powder (30 dose and 15 dose devices respectively) and are capable of holding and delivering two different drug formulations simultaneously (see U.S. Pat. No. 6,561,186). The device uses i-Point™ technology which allows to release medication when predetermined inspiratory flow rate is reached. Adjusting the flow rate (factory setting) can target drug delivery to either lower or upper airways. The device also has an integral dose counter.

Also included in the invention is the Twisthaler® (Schering-Plough) which is a multiple dose device with a nice dose counting feature and is capable of 14-200 actuations (U.S. Pat. No. 5,829,434). The formulation is packaged in a cartridge that contains a desiccant. Products including this DPI device includes Asmanex Twisthaler (mometasone furoate).

Another DPI device which may be used in the methods and compositions of the invention includes SkyeHaler® DPI (SkyePharma) which is a multidose device containing up to 300 individual doses in a single-use, or replaceable cartridge (see U.S. Pat. No. 6,182,655, WO97/20589). The dosing mechanism can handle individual doses from 200 mcg to 5 mg. The device is powered by breath and requires no coordination between breathing and actuation. This DPI is device is included in Foradil Certihaler® (formoterol fumarate).

Also included in the invention is the Novolizer® (Meda AB) which is a refillable, multi-dose, breath activated, dry powder inhaler with dose counter (U.S. Pat. No. 5,840,279, U.S. Pat. No. 6,071,498, WO9700703). The device is used with a refill cartridge containing bulk drug powder for up to 300 single doses. The Novolizer is included in the following: Budesonide 200 μg Novolizer®; Salbutamol 100 μg Novolizer®; Fomoterol Novolizer®; Budesonide Novolizer® 400 μg.

Another DPI device which may be used in the methods and compositions of the invention includes the Blister Inhaler™ (Meda AB), which is a refillable, multi-dose, breath activated, dry powder inhaler with dose counter (U.S. Pat. No. 5,881,719, WO9702061). The device is used with a refill cartridge containing bulk drug powder for up to 300 single doses. The device is able to deliver moisture-sensitive compounds (e.g. proteins and peptides).

Other DPI devices include the SpinHaler® (Aventis and Rhone-Poulenc Rorer; a single dose inhaler that utilizes gelatin capsules to hold micomized drug, including sodium cromoglycate for the treatment of bronchial asthma), the unit dose DPI (Bespak; a device for delivering a single unit dose of a powdered drug, wherein the device contains a powdered drug formulation in a canister/reservoir, inlet valve, a membrane, plunger and a piercing tip; see US2003178440), the DiskHaler® (GlaxoSmithKline; a multidose device (4-8 doses) for local lung delivery—see U.S. Pat. No. 5,035,237), Rotohaler® (GlaxoSmithKline) which is a single use device that utilizes capsules (see U.S. Pat. No. 5,673,686, U.S. Pat. No. 5,881,721); LABHaler® (LAB International; which is a breath-actuated disposable single dose inhalation device for dry powder medication, and is made of one component consisting of an air-duct (air/powder mixer) between a delivery area and the mouthpiece); AirMaX™ (Ivax; a multiple dose reservoir inhaler, where the dose is metered by a small amount of air that is compressed when the user depresses the spring-loaded button; see U.S. Pat. No. 5,503,144); Aerolizer™ (Novartis), which is a single dose dry powder inhaler in which medicament is stored in a capsule and is released by piercing the capsule wall with TEFLON-coated steel pins; see U.S. Pat. No. 6,488,027, U.S. Pat. No. 3,991,761); Rexam DPI (Rexam Pharma; see U.S. Pat. No. 5,651,359 and EP0707862; a single dose, reusable device designed for use with capsules); bead inhaler multiple dose (Valois; WO0035523, U.S. Pat. No. 6,056,169, US2005087188; a multiple dose DPI pulmonary delivery device based on licensed device engine from Elan/Dura/Quadrant); Aspirair® (Ventura; WO 02/089880; a single dose, breath activated DPI that utilizes low pressure air to aerosolize up to 5 mg of dry powder formulation for systemic delivery applications from the lung); and Gyrohaler® (Ventura; GB2407042; a passive disposable DPI containing a strip of blisters with one month's doses for local delivery of drugs to the lung).

Other examples of commercially available dry powder inhalers suitable for use in accordance with the methods herein include the Spinhaler® powder inhaler (Fisons) and the Ventolin® Rotahaler® (GlaxoSmithKline). See also the dry powder delivery devices described in WO 93/00951, WO 96/09085, WO 96/32152, and U.S. Pat. Nos. 5,458,135, 5,785,049, and 5,993,783, herein incorporated by reference.

In one embodiment, the invention provides a dry powder inhaler (DPI) device for pulmonary administration of a TNFα inhibitor to a subject, wherein the DPI device comprises a reservoir comprising an inhalable powder or dry powder composition comprising the TNFα inhibitor, and a means for introducing the inhalable powder or dry powder composition into the subject via inhalation. The invention also provides an inhalable powder which comprises the TNFα inhibitor and is administered to the subject via a dry powder inhaler (DPI).

The DPI device used in the invention may be either a single dose or a multidose inhaler. In addition, the DPI device used in the invention may also be either pre-metered or device-metered.

### Metered Dose Inhaler (MDI) Device

In one embodiment, the TNFα inhibitor, including a TNFα antibody, or antigen-binding portion thereof, is delivered to a subject through metered dose inhaler (MDI) device. An MDI device uses a propellant to deliver reproducible metered drug dose to the lung, and comprises a drug or agent, propellants (e.g. hydrofluoroalkanes (HFA)), surfactants (e.g. phosphatidyl choline, phosphatidyl ethanolamine, phosphatidyl inositol, lysophosphatidyl choline, phosphatidic acid, triglycerides, monogycerides, soy lecithin, fatty acids, and alkyl-polyglycosides), and solvents. An MDI device is often a compact pressurized dispenser, including a canister, metering valve, and spacer. The dose administered by an MDI device is generally in mg and ranges in volume from about 25 to 100 mL. Additionally, MDI devices are advantageous as they are tamper-proof.

Examples of CFC-free MDI products include Albuterol® HFA (Ivax), Atrovent®-HFA (Boehringer-Ingelheim), Proventil®-HFA (3M), Flovent®-HFA (GSK), Qvar® (3M), Ventolin® HFA(GSK), Xopenex® HFA (3M/Sepracor), Salamol Easi-Breathe® CFC-Free (Ivax), Berotec® (Boehringer-Ingelheim), Berodual® (Boehringer-Ingelheim), Intal® Forte (Rhone/Aventis), and Seretide® EvoHaler® (GSK).

Examples of MDI devices include, but are not limited to, the following:

In one embodiment, the invention provides an MDI device for pulmonary administration of a TNFα inhibitor to a subject, wherein the MDI device is an AutoHaler® (3M) (see U.S. Pat. No. 6,120,752) Examples of AutoHaler® devices being used to deliver therapeutic agents include Aerobid® (flunisolide), Alupent® (metaproterenol sulphate), Atrovent®/Atovent®-HFA (ipratropium bromide), Combivent® (albuterol sulfate/ipatropium bromide), MaxAir® AutoHaler® (pirbuterol acetate), Proventil®-HFA (albuterol sulphate), Qvar® (beclomethasone dipropionate) and Xopenex® HFA (levalbuterol hydrochloride)

Another MDI device which may be used in the methods and compositions of the invention includes MD Turbo™ (Accentia Bio) which is a breath-activated accessory device for use with MDIs to improve patient coordination and delivery, which can transform over 90% of dispensed metered-dose inhalers into a breath-activated, dose-counting inhaler. Its features include: i-Point technology (predetermined inspiratory pressure activation) coordinates MDI actuation with the patient's breath; dose-counting mechanism for tracking the number of doses remaining in the inhaler; versatility as it can accept a multiplicity of presently approved MDI products; and ease of use—two-step operation to deliver dose.

In one embodiment, the invention provides an MDI device for pulmonary administration of a TNFα inhibitor to a subject, wherein the MDI device is the WatchHaler® (Activaero GmbH) which is a continuous inhalation flow device for MDIs controlled by a mechanical valve/balloon. The device lowers the inhalation flow rate to improve dosing of aerosols in children. Its features include continuous inhalation flow controlled by a mechanical valve; limitation of inhalation volume by a balloon; high intrathoracic deposition reproducible dosage; purely mechanical driven, no electronics; and visual control of inhalation.

The EZ Spacer® may also be used in the methods and compositions of the invention. The EZ Spacer® (AirPharma) is a portable drug delivery system designed for use with most metered dose inhalers. EZ Spacer® has a visual sign when treatment is completed as the clear reservoir bag collapses when medication has been inhaled. Its features include: collapsible—provides visual cue that the patient is inhaling correctly and that they are receiving their medication; portable & compact—fits easily into a pocket; durable—designed to last at least 1 year; fits all metered dose inhalers; available with or without a mask.

In one embodiment, the Asmair® (Bang and Olufsen Medicom AS) is used in the invention. The Asmair® is an MDI that features and integrated dose-counting device and an assisted firing mechanism, making it easier for patients to use. Its feature also includes a single dose counter.

In one embodiment, the invention includes an Active DPI/MPI device (Bespak) which is a device that employs multiple, carbon fiber brush, setaceous electrodes to disperse powders and aerosols into fine/particles/mists (see WO9419042). As the patient inhales, a 1 to 10 kvolts is passed through the electrodes to disperse the powder/aerosol. A breath sensor comprised of a piezoelectric membrane that flexes in response to a change of air pressure in the passageway and thereby generate a signal representative of inhalation being sensed is employed to initiate the electric discharge. Metering valve for assembly with a pressurized dispensing container, the valve comprising a valve stem co-axially slidable within a valve body defining a metering chamber, inner and outer seals for sealing between the valve body and the valve stem, and a gasket located on the valve body for sealing against a neck portion of a pressurised dispensing container, wherein at least one of the inner seal, outer seal or gasket is formed as a co-moulding with at least a part of the valve body.

In one embodiment, the invention provides an MDI device for pulmonary administration of a TNFα inhibitor to a subject, wherein the MDI device is a device for delivering metered aerosols comprising an active ingredient in solution in a propellant consisting of a hydrofluoroalkane (HFA) (see WO0149350; Chiesi).

Other examples of MDI devices which may be used in the invention include MDI inhalers described in U.S. Pat. No. 6,170,717 (GlaxoSmithKline); EasiBreath® MDI (Ivax; WO193933, U.S. Pat. No. 5,447,150); MDI breath coordinated inhaler and breath actuated inhaler (Kos; CA2298448 and WO2004082633; a breath coordinated inhaler is constructed of molded plastic and designed to accept standard canister cartridges, and a device used in the delivery of biologics such as insulin using HFA propellants); Tempo™ (MAP Pharma; U.S. Pat. No. 6,095,141, U.S. Pat. No. 6,026,808 and U.S. Pat. No. 6,367,471; an MDI which utilizes a standard aerosol MDI canister and metering valve, encased in a compact device that provides an aerosol flow-control chamber and a synchronized triggering mechanism); Xcelovent™ (Meridica/Pfizer; WO9852634; a breath operated device that also has a dose counter feature); and Increased dosage MDI (Nektar WO2004041340; a device capable of delivering 2 mg to 5 mg of a formulated drug using HFA propellants; device utilizes an additional pressure source (pressurizer) to compensate for reduced vapor pressure during actuation which allows for larger doses being effectively aerosolized: and a MDI described in WO03053501 (Vectura; a device that allows the optimization of the output characteristics of drug solution formulations in HFAs by using actuators with laser-drilled orifices of specific dimensions (0.30 mm or less); actuators allow the use of solution formulations with high ethanol content and a high ratio of ethanol to active ingredients and thus, the use of poorly soluble active ingredients in solution formulations and allow the use of solution formulations which are substantially free of low volatility components).

Thus, the invention also includes a metered dose inhaler (MDI) device for pulmonary administration of a TNFα inhibitor to a subject, the MDI device comprising a pressurized canister comprising an aerosol comprising the TNFα inhibitor and a propellant, and a means for introducing the aerosol into the subject via inhalation.

### Nebulizer/Liquid Inhaler

In one embodiment, the TNFα inhibitor, including a TNFα antibody, or antigen-binding portion thereof, is delivered to a subject using a nebulizer or liquid inhaler. Generally, nebulizers use compressed air to deliver medicine as wet aerosol or mist for inhalation, and, therefore, require that the drug be soluble in water. Nebulizer devices can deliver relatively large doses in comparison to MDI or DPI devices, and are especially effective for delivery to the deep lung (peripheral lung region). No propellants are required for nebulizers, which includes jet nebulizers (air-jet nebulizers and liquid-jet nebulizers) and ultrasonic nebulizers.

Examples of nebulizers include Akita™ (Activaero GmbH) (see US2001037806, EP1258264). Akita™ is a table top nebulizer inhalation system (Wt: 7.5 kg, B×W×H: 260×170×270) based on Pari's LC Star that provides full control over patient's breathing pattern. The device can deliver as much as 500 mg drug in solution in less than 10 min with a very high delivery rates to the lung and the lung periphery. 65% of the nebulized particles are below 5 microns and the mass median aerodynamic diameter (MMAD) is 3.8 microns at 1.8 bar. The minimum fill volume is 2 mL and the maximum volume is 8 mL. The inspiratory flow (200 mL/sec) and nebulizer pressure (0.3-1.8 bar) are set by the smart card. The device can be individually adjusted for each patient on the basis of a lung function test.

Another example of a nebulizer which may be used in the methods and compositions of the invention includes the Aeroneb® Go/Pro/Lab nebulizers (AeroGen). The Aeroneb® nebulizer is based on OnQ™ technology, i.e., an electronic micropump (⅜ inch in diameter and wafer-thin) comprised of a unique dome-shaped aperture plate that contains over 1,000 precision-formed tapered holes, surrounded by a vibrational element. Aeroneb® Go is a portable unit for home use, whereas Aeroneb® Pro is a reusable and autoclavable device for use in hospital and ambulatory clinic, and Aeroneb® Lab is a device for use in pre-clinical aerosol research and inhalation studies. The features of the systems include optimization and customization of aerosol droplet size; low-velocity aerosol delivery with a precisely controlled droplet size, aiding targeted drug delivery within the respiratory system; flexibility of dosing; accommodation of a custom single dose ampoule containing a fixed volume of drug in solution or suspension, or commercially available solutions for use in general purpose nebulizers; continuous, breath-activated or programmable; and adaptable to the needs of a broad range of patients, including children and the elderly; single or multi-patient use.

Aerocurrent™ (AerovertRx corp) may also be used in the methods and compositions of the invention (see WO2006006963). This nebulizer is a portable, vibrating mesh nebulizer that features a disposable, pre-filled or user filled drug cartridge.

Staccato™ (Alexza Pharma) may also be used in the methods and compositions of the invention (see WO03095012). The key to Staccato™ technology is vaporization of a drug without thermal degradation, which is achieved by rapidly heating a thin film of the drug. In less than half a second, the drug is heated to a temperature sufficient to convert the solid drug film into a vapor. The inhaler consists of three core components: a heating substrate, a thin film of drug coated on the substrate, and an airway through which the patient inhales. The inhaler is breath-actuated with maximum dose delivered to be 20-25 mg and MMAD in the 1-2 micron range. AERx® (Aradigm) may also be used in the methods and compositions of the invention (see WO9848873, U.S. Pat. No. 5,469,750, U.S. Pat. No. 5,509,404, U.S. Pat. No. 5,522,385, U.S. Pat. No. 5,694,919, U.S. Pat. No. 5,735,263, U.S. Pat. No. 5,855,564). AERx® is a hand held battery operated device which utilizes a piston mechanism to expel formulation from the AERx® Strip. The device monitors patients inspiratory air flow and fires only when optimal breathing pattern is achieved. The device can deliver about 60% of the dose as emitted dose and 50-70% of the emitted dose into deep lung with <25% inter-subject variability.

Another example of a nebulizer device which may also be used in the methods and compositions of the invention includes Respimat® (Boehringer). Respimat® is a multi-dose reservoir system that is primed by twisting the device base, which is compressed a spring and transfers a metered volume of formulation from the drug cartridge to the dosing chamber. When the device is actuated, the spring is released, which forces a micro-piston into the dosing chamber and pushes the solution through a uniblock; the uniblock consists of a filter structure with two fine outlet nozzle channels. The MMAD generated by the Respimat® is 2 um, and the device is suitable for low dose drugs traditionally employed to treat respiratory disorders.

The TNFα inhibitor may also be delivered using the Collegium Nebulizer™ (Collegium Pharma), which is a nebulizer system comprised of drug deposited on membrane. The dosage form is administered to a patient through oral or nasal inhalation using the Collegium Nebulizer after reconstitution with a reconstituting solvent.

Another example of a nebulizer device which may also be used in the methods and compositions of the invention includes the Inspiration® 626 (Respironics). The 626 is a compressor based nebulizer for home care. The 626 delivers a particle size between 0.5 to 5 microns.

Nebulizers which are used in the invention may include Adaptive Aerosol Delivery® technology (Respironics), which delivers precise and reproducible inhaled drug doses to patients regardless of the age, size or variability in breathing patterns of such patients. AAD® systems incorporate electronics and sensors within the handpiece to monitor the patient's breathing pattern by detecting pressure changes during inspiration and expiration. The sensors determine when to pulse the aerosol delivery of medication during the first part of inspiration. Throughout the treatment, the sensors monitor the preceding three breaths and adapt to the patient's inspiratory and expiratory pattern. Because AAD® systems only deliver medication when the patient is breathing through the mouthpiece, these devices allow the patient to take breaks in therapy without medication waste. Examples of AAD® system nebulizers include the HaloLite® AAD®, ProDose® AAD®, and I-Neb® AAD®.

The HaloLite® Adaptive Aerosol Delivery (AAD)® (Respironics) is a pneumatic aerosolisation system powered by a portable compressor. The AAD® technology monitors the patient's breathing pattern (typically every ten milliseconds) and, depending upon the system being used, either releases pulses of aerosolized drug into specific parts of the inhalation, or calculates the dose drawn during inhalation from a “standing aerosol cloud” (see EP 0910421, incorporated by reference herein).

The ProDos AAD® (Respironics) is a nebulizing system controlled by “ProDose Disc™” system. (Respironics). ProDos AAD® is a pneumatic aerosol system powered by a portable compressor, in which the dose to be delivered is controlled by a microchip-containing disc inserted in the system that, among other things, instructs the system as to the dose to deliver. The ProDose Disc™ is a plastic disc containing a microchip, which is inserted into the ProDose AAD® System and instructs it as to what dose to deliver, the number of doses, which may be delivered together with various control data including drug batch code and expiry date (see EP1245244, incorporated by reference herein).

Promixin® can be delivered via Prodose AAD® for management of pseudomonas aeruginosa lung infections, particularly in cystic fibrosis. Promixin® is supplied as a powder for nebulization that is reconstituted prior to use.

The I-neb AAD® is a handheld AAD® system that delivers precise and reproducible drug doses into patients' breathing patterns without the need for a separate compressor (“I-Neb”). The I-neb AAD® is a miniaturized AAD® inhaler based upon a combination of electronic mesh-based aerosolisation technology (Omron) and AAD® technology to control dosing into patients' breathing patterns. The system is approximately the size of a mobile telephone and weighs less than 8 ounces. I-neb AAD® has been used for delivery of Ventavis® (iloprost) (CoTherix/Schering AG).

Another example of a nebulizer which may be used in the methods and compositions of the invention is Aria™ (Chrysalis). Aria is based on a capillary aerosol generation system. The aerosol is formed by pumping the drug formulation through a small, electrically heated capillary. Upon exiting the capillary, the formulation rapidly cooled by ambient air to produce an aerosol with MMAD ranging from 0.5-2.0 um.

In addition the TouchSpray™ nebulizer (Odem) may be used to deliver a TNF inhibitor in accordance with the invention. The TouchSpray™ nebulizer is a hand-held device which uses a perforate membrane, which vibrates at ultrasonic frequencies, in contact with the reservoir fluid, to generate the aerosol cloud. The vibration action draws jets of fluid though the holes in the membrane, breaking the jets into drug cloud. The size of the droplets is controlled by the shape/size of the holes as well as the surface chemistry and composition of the drug solution. This device has been reported to deliver 83% of the metered dose to the deep lung. Details of the TouchSpray™ nebulizer are described in U.S. Pat. No. 6,659,364, incorporated by reference herein.

Additional nebulizers which may be used in the invention include nebulizers which are portable units which maximize aerosol output when the patient inhales and minimize aerosol output when the patient exhales using two one-way valves (see PARI nebulizers (PARI GmbH). Baffles allow particles of optimum size to leave the nebulizer. The result is a high percentage of particles in the respirable range that leads to improved drug delivery to the lungs. Such nebulizers may be designed for specific patient populations, such a patients less than three years of age (PARI BABY™) and nebulizers for older patients (PARI LC PLUS® and PARI LC STAR®).

An additional nebulizer which may be used in the invention is the e-Flow® nebulizer (PARI GmbH) which uses vibrating membrane technology to aerosolize the drug solution, as well as the suspensions or colloidal dispersions (, TouchSpray™; ODEM (United Kingdom)). An e-Flow® nebulizer is capable of handling fluid volumes from 0.5 ml to 5 ml, and can produce aerosols with a very high density of active drug, a precisely defined droplet size, and a high proportion of respirable droplets delivered in the shortest possible amount of time. Drugs which have been delivered using the e-Flow® nebulizer include aztreonam and lidocaine. Additional details regarding the e-Flow® nebulizer are described in U.S. Pat. No. 6,962,151, incorporated by reference herein. Additional nebulizers which may be used in the invention include a Microair® electronic nebulizer (Omron) and a Mystic™ nebulizer (Ventaira). The Microair® nebulizer is extremely small and uses Vibrating Mesh Technology to efficiently deliver solution medications. The Microair device has 7 mL capacity and produces drug particle MMAD size around 5 microns. For additional details regarding the Microair® nebulizer see US patent publication no. 2004045547, incorporated by reference herein. The Mystic™ nebulizer uses strong electric field to break liquid into a spray of nearly monodispersed, charged particles. The Mystic™ system includes a containment unit, a dose metering system, aerosol generation nozzles, and voltage converters which together offer multi-dose or unit-dose delivery options. The Mystic™ device is breath activated, and has been used with Corus 1030™ (lidocaine HCl), Resmycin® (doxorubicin hydrochloride), Acuair (fluticasone propionate), NCE with ViroPharm, and NCE with Pfizer. Additional details regarding the Mystic™ nebulizer may be found in U.S. Pat. No. 6,397,838, incorporated by reference herein.

Thus, in one embodiment, the invention provides a container for use with a nebulizer device for pulmonary administration of a TNFα inhibitor to a subject, the container comprising a propellant-free inhalable solution or suspension comprising the TNFα inhibitor.

The TNFα inhibitor may be administered to a subject via inhalation in accordance with a dosing regimen designed to achieve a therapeutic effect. In one embodiment, a biweekly dosing regimen may be used to treat disorders in which TNFα activity is detrimental using the methods described herein, and are further described in U.S. application Ser. No. 10/163,657. Multiple variable dose methods of treatment can also be used to treat disorders in which TNFα activity is detrimental, and are further described in PCT appln. no. PCT/US05/012007.

### Pharmaceutical Compositions

Antibodies, antibody-portions, and other TNFα inhibitors for use in the methods of the invention, can be incorporated into pharmaceutical compositions suitable for pulmonary administration to a subject.

The compositions for use in the methods and compositions of the invention may be in a variety of forms according to the mode of inhalation and therapeutic application. In one embodiment, the invention provides a pharmaceutical composition comprising a TNFα antibody and a pharmaceutically acceptable carrier, wherein the pharmaceutical composition is suitable for inhalation by a subject. Thus, the TNFα inhibitor is formulated in a pharmaceutical composition suitable for inhalation. Examples of pharmaceutical compositions which are suitable for inhalation include, but are not limited to, an inhalable powder or a dry powder composition, a propellant-containing aerosol, and a propellant-free inhalable solution or suspension. Such pharmaceutical compositions may be administered according to the devices described above. For example, an inhalable powder comprising the TNFα inhibitor may be administered to the subject via a dry powder inhaler (DPI). In another example, a propellant-containing aerosol comprising the TNFα inhibitor may be administered to the subject via a metered dose inhaler (MDI). In yet another example, a propellant-free inhalable solution comprising the TNFα inhibitor may be administered to the subject via a nebulizer. Other suitable preparations include, but are not limited to, mist, vapor, or spray preparations so long as the particles comprising the protein composition are delivered in a size range consistent with that described for the delivery device, e.g., a dry powder form of the pharmaceutical composition.

Thus, a liquid pharmaceutical composition comprising a TNFα antibody, or antigen-binding portion thereof, intended for use in the methods of the present invention may either be used as a liquid solution or suspension in the delivery device or first be processed into a dry powder form using lyophilization or spray-drying techniques well known in the art. Powder comprising a TNFα inhibitor such as a TNFα antibody, may also be prepared using other methods known in the art, including crystallization or precipitation (see, for example, dry powder microspheres (PROMAXX; Baxter) described in U.S. Pat. No. 5,525,519; U.S. Pat. No. 5,599,719; U.S. Pat. No. 5,578,709; U.S. Pat. No. 5,554,730; U.S. Pat. No. 6,090,925; U.S. Pat. No. 5,981,719; U.S. Pat. No. 6,458,387, each of which is incorporated herein by reference).

Where a liquid solution or suspension is used in the delivery device, a nebulizer, a metered dose inhaler, or other suitable delivery device delivers, in a single or multiple fractional dose, by pulmonary inhalation a pharmaceutically effective amount of the composition to the subject's lungs as droplets having the same particle size range noted above for the dry powder form.

Where the liquid pharmaceutical composition is lyophilized prior to use in the delivery methods of the invention, the lyophilized composition may be milled to obtain the finely divided dry powder consisting of particles within the desired size range noted above. Where spray-drying is used to obtain a dry powder form of the liquid pharmaceutical composition, the process is carried out under conditions that result in a substantially amorphous finely divided dry powder consisting of particles within the desired size range noted above. Similarly, if the starting pharmaceutical composition is already in a lyophilized form, the composition can be milled to obtain the dry powder form for subsequent preparation as an aerosol or other preparation suitable for pulmonary inhalation. Where the starting pharmaceutical composition is in its spray-dried form, the composition has preferably been prepared such that it is already in a dry powder form having the appropriate particle size for dispensing as an aqueous or nonaqueous solution or suspension or dry powder form in accordance with the pulmonary administration methods of the invention. For methods of preparing dry powder forms of pharmaceutical compositions, see, for example, WO 96/32149, WO 97/41833, WO 98/29096, and U.S. Pat. Nos. 5,976,574, 5,985,248, and 6,001,336; herein incorporated by reference.

The resulting dry powder form of the composition is then placed within an appropriate delivery device for subsequent preparation as an aerosol or other suitable preparation that is delivered to the subject via pulmonary inhalation. Where the dry powder form of the pharmaceutical composition is to be prepared and dispensed as an aqueous or nonaqueous solution or suspension, a metered-dose inhaler, or other appropriate delivery device is used. A pharmaceutically effective amount of the dry powder form of the composition is administered in an aerosol or other preparation suitable for pulmonary inhalation. The amount of dry powder form of the composition placed within the delivery device is sufficient to allow for delivery of a pharmaceutically effective amount of the composition to the subject by inhalation. Thus, the amount of dry powder form to be placed in the delivery device will compensate for possible losses to the device during storage and delivery of the dry powder form of the composition. Following placement of the dry powder form within a delivery device, the properly sized particles as noted above are suspended in an aerosol propellant. The pressurized nonaqueous suspension is then released from the delivery device into the air passage of the subject while inhaling. The delivery device delivers, in a single or multiple fractional dose, by pulmonary inhalation a pharmaceutically effective amount of the composition to the subject's lungs. The aerosol propellant may be any conventional material employed for this purpose, such as a chlorofluorocarbon, a hydrochloro-fluorocarbon, a hydrofluorocarbon, or a hydrocarbon, including trichlorofluoromethane, dichlorodifluoro-methane, dichlorotetrafluoromethane, dichlorodifluoro-methane, dichlorotetrafluoroethanol, and 1,1,1,2-tetra-fluoroethane, or combinations thereof. A surfactant may be added to the pharmaceutical composition to reduce adhesion of the protein-containing dry powder to the walls of the delivery device from which the aerosol is dispensed. Suitable surfactants for this intended use include, but are not limited to, sorbitan trioleate, soya lecithin, and oleic acid. Devices suitable for pulmonary delivery of a dry powder form of a protein composition as a nonaqueous suspension are commercially available. Examples of such devices include the Ventolin metered-dose inhaler (Glaxo Inc., Research Triangle Park, N.C.) and the Intal Inhaler (Fisons, Corp., Bedford, Mass.). See also the aerosol delivery devices described in U.S. Pat. Nos. 5,522,378, 5,775,320, 5,934,272 and 5,960,792, herein incorporated by reference.

Where the solid or dry powder form of the pharmaceutical composition is to be delivered as a dry powder form, a dry powder inhaler or other appropriate delivery device is preferably used. The dry powder form of the pharmaceutical composition is preferably prepared as a dry powder aerosol by dispersion in a flowing air or other physiologically acceptable gas stream in a conventional manner. Examples of dry powder inhalers suitable for use in accordance with the methods herein are described above.

The dry powder form of the pharmaceutical composition comprising a TNFα inhibitor may be reconstituted to an aqueous solution for subsequent delivery as an aqueous solution aerosol using a nebulizer, a metered dose inhaler, or other suitable delivery device. In the case of a nebulizer, the aqueous solution held within a fluid reservoir is converted into an aqueous spray, only a small portion of which leaves the nebulizer for delivery to the subject at any given time. The remaining spray drains back into a fluid reservoir within the nebulizer, where it is aerosolized again into an aqueous spray. This process is repeated until the fluid reservoir is completely dispensed or until administration of the aerosolized spray is terminated. Examples of nebulizers are described above.

When a pharmaceutical composition comprising a TNFα inhibitor is processed into a solid or dry powder form for subsequent delivery as an aerosol, it may be desirable to have carrier materials present that serve as a bulking agent or stabilizing agent. In this manner, the present invention discloses stabilized lyophilized or spray-dried pharmaceutical compositions comprising a TNFα inhibitor for use in the methods of the present invention. These compositions may further comprise at least one bulking agent, at least one agent in an amount sufficient to stabilize the protein during the drying process, or both. By “stabilized” is intended the TNFα inhibitor thereof retains its monomeric or multimeric form as well as its other key properties of quality, purity, and potency following lyophilization or spray-drying to obtain the solid or dry powder form of the composition.

Preferred carrier materials for use as a bulking agent include glycine, mannitol, alanine, valine, or any combination thereof, most preferably glycine. The bulking agent is present in the formulation in the range of 0% to about 10% (w/v), depending upon the agent used. When the bulking agent is glycine, it is present in the range of about 0% to about 4%, preferably about 0.25% to about 3.5%, more preferably about 0.5% to 3.0%, even more preferably about 1.0% to about 2.5%, most preferably about 2.0%. When the bulking agent is mannitol, it is present in the range of about 0% to about 5.0%, preferably about 1.0% to about 4.5%, more preferably about 2.0% to about 4.0%, most preferably about 4.0%. When the bulking agent is alanine or valine, it is present in the range of about 0% to about 5.0%, preferably about 1.0% to about 4.0%, more preferably about 1.5% to about 3.0%, most preferably about 2.0%.

Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%.

These stabilized lyophilized or spray-dried compositions may optionally comprise methionine, ethylenediaminetetracetic acid (EDTA) or one of its salts such as disodium EDTA or other chelating agent, which protect TNFα inhibitor against methionine oxidation. Methionine is present in the stabilized lyophilized or spray-dried pharmaceutical compositions at a concentration of about 0 to about 10.0 mM, preferably about 1.0 to about 9.0 mM, more preferably about 2.0 to about 8.0 mM, even more preferably about 3.0 to about 7.0 mM, still more preferably about 4.0 to about 6.0 mM, most preferably about 5.0 mM. EDTA is present at a concentration of about 0 to about 10.0 mM, preferably about 0.2 mM to about 8.0 mM, more preferably about 0.5 mM to about 6.0 mM, even more preferably about 0.7 mM to about 4.0 mM, still more preferably about 0.8 mM to about 3.0 mM, even more preferably about 0.9 mM to about 2.0 mM, most preferably about 1.0 mM.

The stabilized lyophilized or spray-dried compositions may be formulated using a buffering agent, which maintains the pH of the pharmaceutical composition within an acceptable range when in a liquid phase, such as during the formulation process or following reconstitution of the dried form of the composition. Preferably the pH is in the range of about pH 4.0 to about pH 8.5, more preferably about pH 4.5 to about pH 7.5, even more preferably about pH 5.0 to about pH 6.5, more preferably still about pH 5.6 to about pH 6.3, and most preferably about pH 5.7 to about pH 6.2. Suitable pH's include about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, up to about 8.5. Most preferably, the pH is about 5.8.

Suitable buffering agents include, but are not limited to, citrate buffer, phosphate buffer, succinate buffer, more particularly a sodium citrate/citric acid. Alternatively imidazole or histidine or other base/acid that maintains pH in the range of about pH 4.0 to about 8.5 can be used. Buffers are chosen such that they are compatible with the drying process and do not affect the quality, purity, potency, and stability of the protein during processing and upon storage.

Any of the pharmaceutical compositions comprising a TNFα inhibitor contemplated for use in the methods of the invention may be formulated with at least one surfactant in an amount sufficient to enhance absorption of the inhaled particles comprising a TNFα inhibitor to obtain an absorbable composition for use in pulmonary inhalation in accordance with the methods described herein.

Any surfactant that enhances absorption of a pharmaceutical composition comprising a TNFα inhibitor thereof in the manner disclosed herein may be used to obtain these absorbable protein-containing pharmaceutical compositions. Surfactants suitable for use in enhancing absorption of the inhaled TNFα inhibitor include, but are not limited to, polyoxyethylene sorbitol esters such as polysorbate 80 (Tween 80) and polysorbate 20 (Tween 20); polyoxypropylene-polyoxyethylene esters such as Poloxamer 188; polyoxyethylene alcohols such as Brij 35; a mixture of polysorbate surfactants with phospholipids such as phosphatidylcholine and derivatives (dipalmitoyl, dioleoyl, dimyristyl, or mixed derivatives such as 1-palmitoyl, 2-olcoyl, etc.), dimyristolglycerol and other members of the phospholipid glycerol series; lysophosphatidylcholine and derivatives thereof; mixtures of polysorbates with lysolecithin or cholesterol; a mixture of polysorbate surfactants with sorbitan surfactants (such as sorbitan monoleate, dioleate, trioleate or others from this class); poloxamer surfactants; bile salts and their derivatives such as sodium cholate, sodium deoxycholate, sodium glycodeoxycholate, sodium taurocholate, etc.; mixed micelles of TNFα inhibitor with bile salts and phospholipids; Brij surfactants (such as Brij 35-PEG923) lauryl alcohol, etc.). The amount of surfactant to be added is in the range of about 0.005% to about 1.0% (w/v), preferably about 0.005% to about 0.5%, more preferably about 0.01% to about 0.4%, even more preferably about 0.03% to about 0.3%, most preferably about 0.05% to about 0.2%.

The pharmaceutical composition of the invention may include a suitable dosage according to the disorder being treated. In one embodiment, the pharmaceutical composition of the invention comprises a dose of about 40 mg of a TNFα antibody, or antigen-binding portion thereof. Alternatively, the pharmaceutical composition of the invention comprises a dose of about 40-160 mg of the TNFα antibody, or antigen-binding portion thereof. In another embodiment, the pharmaceutical composition comprises a dose over 160 mg.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Therapeutic compositions typically must be sterile and stable under the conditions of manufacture and storage. The composition can be formulated as a solution, microemulsion, dispersion, liposome, or other ordered structure suitable to high drug concentration. Sterile inhalable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. The proper fluidity of a solution can be maintained, for example, by the use of a coating such as lecithin, by the maintenance of the required particle size in the case of dispersion and by the use of surfactants. Prolonged absorption of inhalable compositions can be brought about by including in the composition an agent that delays absorption, for example, monostearate salts and gelatin.

In one embodiment, the antibody or antibody portion for use in the methods of the invention is incorporated into a pharmaceutical formulation as described in PCT/IB03/04502 and U.S. Appln. No. 20040033228, incorporated by reference herein. This formulation includes a concentration 50 mg/ml of the antibody D2E7 (adalimumab).

Supplementary active compounds can also be incorporated into the compositions for pulmonary delivery. In certain embodiments, an antibody or antibody portion for use in the methods of the invention is coformulated with and/or coadministered with one or more additional therapeutic agents. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The invention also pertains to packaged pharmaceutical compositions or kits for pulmonary administration of a TNF inhibitor, e.g., antibodies. In one embodiment of the invention, the kit comprises a TNFα inhibitor, such as an antibody, and instructions for pulmonary administration of the TNFα inhibitor, wherein the TNFα inhibitor is in a formulation suitable for inhalation. The instructions may describe when, e.g., at week 0, week 2, week 4, etc., the different doses of TNFα inhibitor shall be administered via inhalation to a subject for treatment.

Another aspect of the invention pertains to kits containing a pharmaceutical composition comprising a TNFα inhibitor, such as an antibody, and a pharmaceutically acceptable carrier, and one or more pharmaceutical compositions each comprising an additional therapeutic agent, and a pharmaceutically acceptable carrier.

The package or kit alternatively can contain the TNFα inhibitor and it can be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### III. TNF Inhibitors

A TNFα inhibitor which is used in the methods and compositions of the invention includes any agent which interferes with TNFα activity. In a preferred embodiment, the TNFα inhibitor can neutralize TNFα activity, particularly detrimental TNFα activity which is associated with Crohn's disease, RA, PsA, JRA, AS, and psoriasis, and related complications and symptoms.

In one embodiment, the TNFα inhibitor used in the invention is a TNFα antibody (also referred to herein as an anti-TNFα antibody), or an antigen-binding fragment thereof, including chimeric, humanized, and human antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein.

Other examples of TNFα inhibitors which may be used in the methods and compositions of the invention include etanercept (Enbrel®, described in WO 91/03553 and WO 09/406,476), soluble TNF receptor Type I, a pegylated soluble TNF receptor Type I (PEGs TNF-R1), p55TNFR1gG (Lenercept), and recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFα inhibitor” excludes adalimumab and infliximab.

In one embodiment, the term “TNFα inhibitor” excludes etanercept, and, optionally, adalimumab, infliximab, and adalimumab and infliximab.

In one embodiment, the term “TNFα antibody” excludes infliximab. In one embodiment, the term “TNFα antibody” excludes adalimumab. In another embodiment, the term “TNFα antibody” excludes adalimumab and infliximab.

In one embodiment, the invention features an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein. The methods of the invention may also be performed using chimeric and humanized murine anti-hTNFα antibodies which have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

In one embodiment, the method of the invention includes pulmonary administration of D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity. In one embodiment, the invention provides treatment with an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to pulmonary administration of human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the K_{off}. Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the K_{off}. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the K_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a K_{off} of 5×10⁻⁴ s⁻¹ or less. Even more preferably, the antibody, or antigen-binding portion thereof, dissociates from human TNFα with a K_{off} of 1×10⁻⁴ s⁻¹ or less.

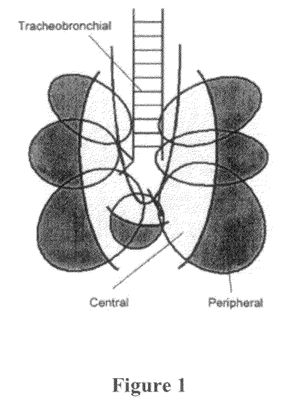

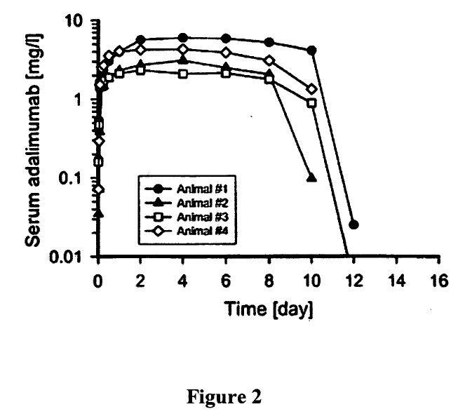

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the V_{κ}I human germline family, more preferably from the A20 human germline Vk gene and most preferably from the D2E7 VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The TNFα antibody used in the methods and compositions of the invention may be used for pulmonary administration. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used for pulmonary administration. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity. For example, the constant regions of the TNFα antibody, or antigen-binding portion thereof, used in the invention for pulmonary administration may be modified such that the antibody has decreased binding to phagocytic receptors expressed on alveolar macrophage.

In another example, the TNFα antibody, or antigen-binding portion thereof, used in the invention for pulmonary administration may be administered in combination with an agent that binds FcR but not FcRn. Such a combination therapy would increase the bioavailability of the TNFα antibody, or antigen-binding portion thereof, by virtue of saturating the FcR pathway.

In still another embodiment, the invention includes pulmonary administration of a modified TNFα antibody, or antigen-binding portion thereof, having enhanced binding to a neonatal Fc receptor (FcRN). Modifications to increase binding to neonatal FcRn may include conjugating the TNFα antibody to a compound which increases transport of the TNFα antibody from the lung epithelium of a subject to the bloodstream of the subject. Additional modifications may also include mutating the TNFα antibody, or antigen-binding portion thereof, wherein the TNFα antibody comprises mutations and/or deletions within the Fc domain which increase the binding affinity of the TNFα antibody to FcRn. Examples of positions within the antibody which may be modified include, but are not limited to, at least one mutation within the Fc domain at an amino acid position selected from the group consisting of 238, 256, 307, 311, 312, 380, and 382 (Shields et al. (2001) J Biol Chem 276:6591).

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V₇₈ Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the V_{H}3 family of human germline VH genes is amplified by standard PCR. Most preferably, the DP-31 VH germline sequence is amplified. To obtain a DNA fragment encoding the light chain variable region of D2E7, or a D2E7-related antibody, a member of the V_{κ}I family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated gemiline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of proteins oflmmunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schafflier et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

Recombinant human antibodies of the invention in addition to D2E7 or an antigen binding portion thereof, or D2E7-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS89:3576-3580; Garrard et al. (1991) Bio/Technology 9:1373-1377; Hoogenboom et al. (1991) NucAcid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J. 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

### IV. Disorders for Treatment with Invention

As used herein, the term “a disorder in which TNFα activity is detrimental” is intended to include diseases and other disorders in which the presence of TNFα in a subject suffering from the disorder has been shown to be or is suspected of being either responsible for the pathophysiology of the disorder or a factor that contributes to a worsening of the disorder. Accordingly, a disorder in which TNFα activity is detrimental is a disorder in which inhibition of TNFα activity is expected to alleviate the symptoms and/or progression of the disorder. Such disorders may be evidenced, for example, by an increase in the concentration of TNFα in a biological fluid of a subject suffering from the disorder (e.g., an increase in the concentration of TNFα in serum, plasma, synovial fluid, etc. of the subject), which can be detected, for example, using an anti-TNFα antibody as described above. There are numerous examples of disorders in which TNFα activity is detrimental, including, but not limited to, an autoimmune disorder, e.g., rheumatoid arthritis (RA) or juvenile rheumatoid arthritis (JRA), a spondyloarthropathy, e.g., ankylosing spondylitis (AS) or psoriatic arthritis (PsA), an intestinal disorder, e.g., Crohn's, a skin disorder, e.g., psoriasis, and a pulmonary disorder, e.g., COPD or asthma.

Additional details regarding TNF disorders are described below.

### A. Autoimmune Diseases

In one embodiment, the invention includes treatment of an autoimmune disease. TNFα antibodies, such as adalimumab, may be used to treat autoimmune diseases. Examples of such autoimmune conditions include rheumatoid arthritis, rheumatoid spondylitis, osteoarthritis and gouty arthritis, allergy, multiple sclerosis, autoimmune diabetes, autoimmune uveitis and nephrotic syndrome. Other examples of autoimmune conditions include multisystem autoimmune diseases and autoimmune hearing loss. Other examples of autoimmune disease are described in U.S. application Ser. No. 10/622,932, incorporated by reference herein.

### Rheumatoid Arthritis

TNFα has been implicated in activating tissue inflammation and causing joint destruction in rheumatoid arthritis (see e.g., Moeller, A., et al. (1990) Cytokine 2:162-169; U.S. Pat. No. 5,231,024 to Moeller et al.; European Patent Publication No. 260 610 B1 by Moeller, A.; Tracey and Cerami, supra; Arend, W. P. and Dayer, J-M. (1995) Arth. Rheum. 38:151-160; Fava, R. A., et al. (1993) Clin. Exp. Immunol. 94:261-266).

### Juvenile Rheumatoid Arthritis

Tumor necrosis factor has been implicated in the pathophysiology of juvenile arthritis, including juvenile rheumatoid arthritis (Grom et al. (1996) Arthritis Rheum. 39:1703; Mangge et al. (1995) Arthritis Rheum. 8:211). In one embodiment, the TNFα antibody of the invention is used to treat juvenile rheumatoid arthritis. The term “juvenile rheumatoid arthritis” or “JRA” as used herein refers to a chronic, inflammatory disease which occurs before age 16 that may cause joint or connective tissue damage. JRA is also referred to as juvenile chronic polyarthritis and Still's disease. JRA causes joint inflammation and stiffness for more than 6 weeks in a child of 16 years of age or less. Inflammation causes redness, swelling, warmth, and soreness in the joints. Any joint can be affected and inflammation may limit the mobility of affected joints. One type of JRA can also affect the internal organs.

JRA is often classified into three types by the number of joints involved, the symptoms, and the presence or absence of certain antibodies found by a blood test. These classifications help the physician determine how the disease will progress and whether the internal organs or skin is affected. The classifications of JRA include the following:

a. Pauciarticular JRA, wherein the patient has four or fewer joints are affected. Pauciarticular is the most common form of JRA, and typically affects large joints, such as the knees.

b. Polyarticular HRA, wherein five or more joints are affected. The small joints, such as those in the hands and feet, are most commonly involved, but the disease may also affect large joints.

c. Systemic JRA is characterized by joint swelling, fever, a light skin rash, and may also affect internal organs such as the heart, liver, spleen, and lymph nodes. Systemic JRA is also referred to as it Still's disease. A small percentage of these children develop arthritis in many joints and can have severe arthritis that continues into adulthood.

### B. Spondyloarthropathies

In one embodiment, the invention includes treatment of a spondylarthopathy. As used herein, the term “spondyloarthropathy” or “spondyloarthropathies” is used to refer to any one of several diseases affecting the joints of the spine, wherein such diseases share common clinical, radiological, and histological features. A number of spondyloarthropathies share genetic characteristics, i.e. they are associated with the HLA-B27 allele. In one embodiment, the term spondyloarthropathy is used to refer to any one of several diseases affecting the joints of the spine, excluding ankylosing spondylitis, wherein such diseases share common clinical, radiological, and histological features. Examples of spondyloarthropathies include ankylosing spondylitis, psoriatic arthritis/spondylitis, enteropathic arthritis, reactive arthritis or Reiter's syndrome, and undifferentiated spondyloarthropathies. Examples of animal models used to study spondyloarthropathies include ank/ank transgenic mice, HLA-B27 transgenic rats (see Taurog et al. (1998) The Spondylarthritides. Oxford:Oxford University Press).

Examples of subjects who are at risk of having spondyloarthropathies include humans suffering from arthritis. Spondyloarthropathies can be associated with forms of arthritis, including rheumatoid arthritis. In one embodiment of the invention, a TNFα inhibitor is used to treat a subject who suffers from a spondyloarthropathy through pulmonary administration of the TNFα inhibitor. Examples of spondyloarthropathies which can be treated with a TNFα inhibitor are described below:

### Ankylosing Spondylitis (AS)

In one embodiment, the invention includes treatment ankylosing spondylitis using a TNFα inhibitor, e.g., TNFα antibody, or antigen-binding portion thereof. Tumor necrosis factor has been implicated in the pathophysiology of ankylosing spondylitis (see Verjans et al. (1991) Arthritis Rheum. 34:486; Verjans et al. (1994) Clin Exp Immunol. 97:45; Kaijtzel et al. (1999) Hum Immunol. 60:140). Ankylosing spondylitis (AS) is an inflammatory disorder involving inflammation of one or more vertebrae. AS is a chronic inflammatory disease that affects the axial skeleton and/or peripheral joints, including joints between the vertebrae of the spine and sacroiliac joints and the joints between the spine and the pelvis. AS can eventually cause the affected vertebrae to fuse or grow together. Spondyarthropathies, including AS, can be associated with psoriatic arthritis (PsA) and/or inflammatory bowel disease (IBD), including ulcerative colitis and Crohn's disease.

Early manifestations of AS can be determined by radiographic tests, including CT scans and MRI scans. Early manifestations of AS often include scroiliitis and changes in the sacroliac joints as evidenced by the blurring of the cortical margins of the subchrondral bone, followed by erosions and sclerosis. Fatigue has also been noted as a common symptom of AS (Duffy et al. (2002) ACR 66th Annual Scientific Meeting Abstract).

### Psoriatic Arthritis

In one embodiment, the invention includes treatment psoriatic arthritis using a TNFα inhibitor, e.g., a TNFα antibody, or antigen-binding portion thereof. Tumor necrosis factor has been implicated in the pathophysiology of psoriatic arthritis (PsA) (Partsch et al. (1998) Ann Rheum Dis. 57:691; Ritchlin et al. (1998) J Rheumatol. 25:1544). As referred to herein, psoriatic TNFα has been implicated in activating tissue inflammation and causing joint destruction in rheumatoid arthritis (see e.g., Moeller, A., et al. (1990) Cytokine 2:162-169; U.S. Pat. No. 5,231,024 to Moeller et al.; European Patent Publication No. 260 610 B1 by Moeller, A.; Tracey and Cerami, supra; Arend, W. P. and Dayer, J-M. (1995) Arth. Rheum. 38:151-160; Fava, R. A., et al. (1993) Clin. Exp. Immunol. 94:261-266). TNFα also has been implicated in promoting the death of islet cells and in mediating insulin resistance in diabetes (see e.g., Tracey and Cerami, supra; PCT Publication No. WO 94/08609). TNFα also has been implicated in mediating cytotoxicity to oligodendrocytes and induction of inflammatory plaques in multiple sclerosis (see e.g., Tracey and Cerami, supra). Chimeric and humanized murine anti-hTNFα antibodies have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

Psoriatic arthritis refers to chronic inflammatory arthritis which is associated with psoriasis, a common chronic skin condition that causes red patches on the body. About 1 in 20 individuals with psoriasis will develop arthritis along with the skin condition, and in about 75% of cases, psoriasis precedes the arthritis. PsA exhibits itself in a variety of ways, ranging from mild to severe arthritis, wherein the arthritis usually affects the fingers and the spine. When the spine is affected, the symptoms are similar to those of ankylosing spondylitis, as described above. A TNFα antibody, or antigen-binding fragment thereof, can be used for treatment of PsA.

PsA is sometimes associated with arthritis mutilans. Arthritis mutilans refers to a disorder which is characterized by excessive bone erosion resulting in a gross, erosive deformity which mutilates the joint.

Characteristic radiographic features of PsA include joint erosions, joint space narrowing, bony proliferation including periarticular and shaft periostitis, osteolysis including “pencil in cup” deformity and acro-osteolysis, ankylosis, spur formation, and spondylitis (Wassenberg et al. (2001) Z Rheumatol 60:156). Unlike rheumatoid arthritis (RA), joint involvement in PsA is often asymmetrical and may be oligoarticular; osteoporosis is atypical. Although erosive changes in early PsA are marginal as in RA, they become irregular and ill defined with disease progression because of periosteal bone formation adjacent to the erosions. In severe cases, erosive changes may progress to development of pencil in cup deformity or gross osteolysis (Gold et al. (1988) Radiol Clin North Am 26:1195; Resnick et al. (1977)) J Can Assoc Radiol 28:187). Asymmetrical erosions may be visible radiographically in the carpus and in the metacarpophalangeal (MCP), proximal interphalangeal (PIP), and distal interphalangeal (DIP) joints of the hands, but the DIP joints are often the first to be affected. Abnormalities are seen in the phalangeal tufts and at the sites of attachments of tendons and ligaments to the bone. The presence of DIP erosive changes may provide both sensitive and specific radiographic findings to support the diagnosis of PsA. Also, the hands tend to be involved much more frequently than the feet with a ratio of nearly 2:1.

Other examples of spondyloarthropathies are described in U.S. application Ser. No. 10/622,932, incorporated by reference herein.

### C. Skin and Nail Disorders

In one embodiment, the invention includes treatment skin and nail disorders. As used herein, the term “skin and nail disorder in which TNFα activity is detrimental” is intended to include skin and/or nail disorders and other disorders in which the presence of TNFα in a subject suffering from the disorder has been shown to be or is suspected of being either responsible for the pathophysiology of the disorder or a factor that contributes to a worsening of the disorder, e.g., psoriasis. Accordingly, skin and nail disorders in which TNFα activity is detrimental are disorders in which inhibition of TNFα activity is expected to alleviate the symptoms and/or progression of the disorder. The use of the antibodies, antibody portions, and other TNFα inhibitors for the treatment of specific skin and nail disorders is discussed further below. In certain embodiments, the antibody, antibody portion, or other TNFα inhibitor of the invention is administered to the subject in combination with another therapeutic agent, as described below. In one embodiment, a TNFα antibody is administered to the subject in combination with another therapeutic agent for the treatment of psoriasis.

### Psoriasis

Tumor necrosis factor has been implicated in the pathophysiology of psoriasis (Takematsu et al. (1989) Arch Dermatol Res. 281:398; Victor and Gottlieb (2002) J Drugs Dermatol. 1 (3):264). Psoriasis is described as a skin inflammation (irritation and redness) characterized by frequent episodes of redness, itching, and thick, dry, silvery scales on the skin. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the epidermis and polymorphonuclear leukocyte and lymphocyte infiltration into the epidermis layer resulting in an increase in the basal cell cycle. Psoriasis often involves the nails, which frequently exhibit pitting, separation of the nail, thickening, and discoloration. Psoriasis is often associated with other inflammatory disorders, for example arthritis, including rheumatoid arthritis, inflammatory bowel disease (IBD), and Crohn's disease.

Evidence of psoriasis is most commonly seen on the trunk, elbows, knees, scalp, skin folds, or fingernails, but it may affect any or all parts of the skin. Normally, it takes about a month for new skin cells to move up from the lower layers to the surface. In psoriasis, this process takes only a few days, resulting in a build-up of dead skin cells and formation of thick scales. Symptoms of psoriasis include: skin patches, that are dry or red, covered with silvery scales, raised patches of skin, accompanied by red borders, that may crack and become painful, and that are usually lovated on the elbows, knees, trunk, scalp, and hands; skin lesions, including pustules, cracking of the skin, and skin redness; joint pain or aching which may be associated with of arthritis, e.g., psoriatic arthritis.

Treatment for psoriasis often includes a topical corticosteroids, vitamin D analogs, and topical or oral retinoids, or combinations thereof. In one embodiment, the TNFα inhibitor of the invention is administered in combination with or the presence of one of these common treatments. Additional therapeutic agents which can also be combined with the TNFα inhibitor for treatment of psoriasis are described in more detail below.

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

In one embodiment of the invention, a TNFα inhibitor is used to treat psoriasis, including chronic plaque psoriasis, guttate psoriasis, inverse psoriasis, pustular psoriasis, pemphigus vulgaris, erythrodermic psoriasis, psoriasis associated with inflammatory bowel disease (IBD), and psoriasis associated with rheumatoid arthritis (RA). Specific types of psoriasis included in the treatment methods of the invention include chronic plaque psoriasis, guttate psoriasis, inverse psoriasis, and pustular psoriasis. Other examples of psoriasis and other types of skin and nail disorders are described in U.S. application Ser. No. 10/622,932, incorporated by reference herein.

### D. Pulmonary Disorders

In one embodiment, the invention provides a method of treating a pulmonary disorder in a subject comprising pulmonary delivery of a TNFα inhibitor to the subject, wherein the pulmonary administration comprises local delivery of the TNFα inhibitor to the lung(s) of the subject. Examples of pulmonary disorders which may be treated according to local delivery methods of the invention include, but are not limited to, COPD and asthma. The term “local” therefore is used herein with respect to the lung.

TNFα has been implicated in the pathophysiology of a wide variety of pulmonary disorders, including pulmonary disorders such as idiopathic interstitial lung disease and chronic obstructive airway disorders (see e.g., Piquet P F et al. (1989) J Exp Med. 170:655-63; Whyte M, et al. (2000) Am J Respir Crit. Care Med. 162:755-8; Anticevich S Z, et al. (1995) Eur J Pharmacol. 284:221-5). The invention provides methods for TNFα activity in a subject suffering from such a pulmonary disorder, which method comprises administering to the subject an antibody, antibody portion, or other TNFα inhibitor such that TNFα activity in the subject suffering from idiopathic interstitial lung disease or a chronic obstructive airway disorder is inhibited. Examples of idiopathic interstitial lung diseases and chronic obstructive airway disorders in which TNFα activity is detrimental are discussed further below.

### 1. Idiopathic Interstitial Lung Disease

In one embodiment, the TNFα antibody of the invention is used to treat subjects who have an idiopathic interstitial lung disease. Idiopathic interstitial lung diseases affect the lungs in three ways: first, the lung tissue is damaged in some known or unknown way; second, the walls of the air sacs in the lung become inflamed; and finally, scarring (or fibrosis) begins in the interstitium (or tissue between the air sacs), and the lung becomes stiff. Examples of idiopathic interstitial lung diseases are described below.

a. Idiopathic Pulmonary Fibrosis (IPF)

Tumor necrosis factor has been implicated in the pathophysiology of idiopathic pulmonary fibrosis (IPF) (see Piquet P F, et al. (1989) J Exp Med. 170:655-63; Whyte M, et al. (2000) Am J Respir Crit. Care Med 162:755-8; Corbett E L, et al. (2002) Am J Respir Crit. Care Med. 165:690-3). For example, it has been found that IPF patients have increased levels of TNF expression in macrophages and in type II epithelial cells (Piquet et al. (1993) Am J Pathol 143:651; Nash et al. (1993) Histopathology 22:343; Zhang et al. (1993) J Immunol 150:4188). Certain genetic polymorphisms are also associated with increased TNF expression, and are implicated in playing a role in IPF and silicosis (Whyte et al., supra; Corbett E L, et al., supra).

The term “idiopathic pulmonary fibrosis” or “IPF” refers to a group of disorders characterized by inflammation and eventually scarring of the deep lung tissues, leading to shortness of breath. The scarring of the alveoli (air sacs) and their supporting structures (the interstitium) in IPF eventually leads to a loss of the functional alveolar units and a reduction of the transfer of oxygen from air to blood. IPF is also referred to as diffuse parenchymal lung disease; alveolitis; cryptogenic fibrosing alveolitis (CFA); idiopathic pulmonary pneumonitis (IPP); and usual interstitial pneumonitis (UIP). IPF is often used synonymously with UIP (“IPF/UIP”) because UIP is the most common cellular pattern seen in the pathologic diagnosis of IPF.

Patients with IPF often exhibit certain symptoms, including a dry cough, chest pain, and/or shortness of breath. Commonly used drugs for the treatment of IPF are prednisone and cytoxan, although only a fraction of patients improve with continued use of these drugs (American Thoracic Society (2000) Am. J. Respir. Crit. Care Med. 161:646). Oxygen administration and transplantation of the lung are other choices for treatment. In one embodiment, the TNFα antibody of the invention is administered to the subject in combination with another therapeutic agent, for example oxygen, for the treatment of idiopathic pulmonary fibrosis.

### 2. Chronic Obstructive Airway Disorder

In one embodiment, the TNFα antibody of the invention is used to treat a subject who has a chronic obstructive airflow disorder. In these diseases, airflow obstruction may be chronic and persistent or episodic and recurrent. Airflow obstruction is usually determined by forced expiratory spirometry, which is the recording of exhaled volume against time during a maximal expiration. In a subject who does not have an obstructed airflow, a full forced expiration usually takes between 3 and 4 seconds. In a patient with chronic obstructive airflow disorder, wherein airflow is obstructed, it usually takes up to 15 to 20 seconds and may be limited by breath-holding time. The normal forced expiratory volume in the first second of expiration (FEV₁) is easily measured and accurately predicted on the basis of age, sex, and height. The ratio of FEV₁ to forced vital capacity (FEV₁/FVC) normally exceeds 0.75. Recording airflow against volume during forced expiration and a subsequent forced inspiration—the flow-volume loop—is also useful, mainly for distinguishing upper from lower airway narrowing. Examples of chronic obstructive airway disorders are described below.

a. Asthma

Tumor necrosis factor has been implicated in the pathophysiology of asthma, (Anticevich S Z, et al. (1995) Eur J Pharmacol. 284:221-5; Thomas P S, et al. 1995. Am J Respir Crit. Care Med. 152:76-80; Thomas P S, Heywood G. (2002) Thorax. 57:774-8). For example, acute asthma attacks have been found to be associated with pulmonary neutrophilia and elevated BAL TNF levels (Ordonez C L. et al. (2000) Am J Respir Crit. Care Med 161:1185). It has been found that the severity of asthma symptoms correlates with endotoxin levels in house dust. In rats, anti-TNF antibodies reduced endotoxin-induced airway changes (Kips et al. (1992) Am Rev Respir Dis 145:332).

The term “asthma” as used herein, refers to a disorder in which inflammation of the airways causes airflow into and out of the lungs to be restricted. Asthma is also referred to as bronchial asthma, exercise induced asthma—bronchial, and reactive airways disease (RAD). In some instances, asthma is associated with allergies and/or is familial. Asthma includes a condition which is characterized by widespread fluctuations in the diameter or caliber of bronchial airways over short periods of time, resulting in changes in lung function. The resulting increased resistance to air flow produces symptoms in the affected subject, including breathlessness (dyspnea), chest constriction or “tightness,” and wheezing.

Patients with asthma are characterized according to NIH guidelines, are described as mild intermittent, mild persistent, moderate persistent, and severe persistent (see NAEPP Expert Panel Report Guidelines for the Diagnosis and Management of Asthma-Update on Selected Topics 2002. JACI-2002; 110: S141-S209; Guidelines for the Diagnosis and Management of Asthma. NIH Publication 97-4051, July 1997). Patients diagnosed with moderate persistent asthma are often treated with inhaled corticosteroids. Patients diagnosed with severe persistent asthma are often treated with high dose inhaled corticosteroids and p.o. corticosteroids.

b. Chronic Obstructive Pulmonary Disease (COPD)

Tumor necrosis factor has been implicated in the pathophysiology of chronic obstructive pulmonary disease, (Keatings V M. (2000) Chest. 118:971-5; Sakao S, et al. (2001) Am J Respir Crit. Care Med. 163:420-22; Sakao S, et al. (2002) Chest. 122:416-20). The term “chronic obstructive pulmonary disease” or “COPD” as used interchangeably herein, refers to a group of lung diseases characterized by limited airflow with variable degrees of air sack enlargement and lung tissue destruction. The term COPD includes chronic bronchitis (mucous hypersecretion with goblet cell submucosal gland hyperplasia), chronic obstructive bronchitis, or emphysema (destruction of airway parenchyma), or combinations of these conditions. Emphysema and chronic bronchitis are the most common forms of chronic obstructive pulmonary disease. COPD is defined by irreversible airflow obstruction.

In COPD, chronic inflammation leads to fixed narrowing of small airways and lung parenchyma and alveolar wall destruction (emphysema). This is characterized by increased numbers of alveolar macrophages, neutrophils, and cytotoxic T lymphocytes, and the release of multiple inflammatory mediators (lipids, chemokines, cytokines, growth factors). This inflammation leads to fibrosis with a narrowing of the small airways and lung parenchymal destruction. There is also a high level of oxidative stress, which may amplify this inflammation.

### V. Additional Therapeutic Agents

A TNFα inhibitor, such as, but not limited to, an antibody, or antigen-binding portion thereof, may be administered through pulmonary delivery in combination with additional therapeutic agents known to be effective at acute management of subjects with a disorder in which TNFα activity is detrimental, including, but not limited to RA, AS, PsA, JRA, psoriasis, and asthma.

TNFα antibodies, or antigen binding portions thereof, may be used alone or in combination to treat such diseases. It should be understood that the antibodies may be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

Binding proteins described herein may be used in combination with additional therapeutic agents such as a Disease Modifying Anti-Rheumatic Drug (DMARD) or a Nonsteroidal Antiinflammatory Drug (NSAID) or a steroid or any combination thereof. Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone; the well known side effects of steroid use can be reduced or even eliminated by tapering the steroid dose required when treating patients in combination with the anti-TNFα antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists such as soluble p55 or p75 TNF receptors, derivatives, thereof, (p75TNFR1gG (Enbrel™) or p55TNFR1gG (Lenercept), chimeric, humanized or human TNF antibodies, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (Humira® Abbott Laboratories, a human anti-TNF mAb, described in U.S. Pat. No. 6,090,382 as D2E7). Additional TNF antibodies which can be used in the invention are described in U.S. Pat. Nos. 6,593,458; 6,498,237; 6,451,983; and 6,448,380, each of which is incorporated by reference herein. Other combinations including TNFα converting enzyme (TACE) inhibitors; IL-1 inhibitors (Interleukin-1-converting enzyme inhibitors, IL-1RA etc.) may be effective for the same reason. Other preferred combinations include Interleukin 11. Yet another preferred combination are other key players of the autoimmune response which may act parallel to, dependent on or in concert with TNFα function; especially preferred are IL-18 antagonists including IL-18 antibodies or soluble IL-18 receptors, or IL-18 binding proteins. It has been shown that TNFα and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNF [symbol] or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram, and Actemrar™ (tocilizumab) humanized MAb against interleukin-6 (IL-6) receptor. Preferred combinations include methotrexate or leflunomide and in moderate or severe rheumatoid arthritis cases, cyclosporine.

Nonlimiting additional agents which can also be used in combination with an TNFα antibody, or antigen-binding portion thereof, to treat rheumatoid arthritis include, but are not limited to, the following: non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2/infliximab (chimeric anti-TNFα antibody; Centocor); 75 kdTNFR-IgG/etanercept (75 kD TNF receptor-IgG fusion protein; Immunex; see e.g., Arthritis & Rheumatism (1994) Vol. 37, S295; J. Invest. Med. (1996) Vol. 44, 235A); 55 kdTNF-IgG (55 kD TNF receptor-IgG fusion protein; Hoffmann-LaRoche); IDEC-CE9.1/SB 210396 (non-depleting primatized anti-CD4 antibody; IDEC/SmithKline; see e.g., Arthritis & Rheumatism (1995) Vol. 38, S185); DAB 486-IL-2 and/or DAB 389-IL-2 (IL-2 fusion proteins; Seragen; see e.g., Arthritis & Rheumatism (1993) Vol. 36, 1223); Anti-Tac (humanized anti-IL-2Rα; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-1RA (IL-1 receptor antagonist; Synergen/Amgen); anakinra/Kineret® (Amgen); TNF-bp/s-TNF (soluble TNF binding protein; see e.g., Arthritis & Rheumatism (1996) Vol. 39, No. 9 (supplement), S284; Amer. J. Physiol.—Heart and Circulatory Physiology (1995) Vol. 268, pp. 37-42); R973401 (phosphodiesterase Type IV inhibitor; see e.g., Arthritis & Rheumatism (1996) Vol. 39, No. 9 (supplement), S282); MK-966 (COX-2 Inhibitor; see e.g., Arthritis & Rheumatism (1996) Vol. 39, No. 9 (supplement), S81); Iloprost (see e.g., Arthritis & Rheumatism (1996) Vol. 39, No. 9 (supplement), S82); methotrexate; thalidomide (see e.g., Arthritis & Rheumatism (1996) Vol. 39, No. 9 (supplement), S282) and thalidomide-related drugs (e.g., Celgen); leflunomide (anti-inflammatory and cytokine inhibitor; see e.g., Arthritis & Rheumatism (1996) Vol. 39, No. 9 (supplement), S131; Inflammation Research (1996) Vol. 45, pp. 103-107); tranexamic acid (inhibitor of plasminogen activation; see e.g., Arthritis & Rheumatism (1996) Vol. 39, No. 9 (supplement), S284); T-614 (cytokine inhibitor; see e.g., Arthritis & Rheumatism (1996) Vol. 39, No. 9 (supplement), S282); prostaglandin E1 (see e.g., Arthritis & Rheumatism (1996) Vol. 39, No. 9 (supplement), S282); Tenidap (non-steroidal anti-inflammatory drug; see e.g., Arthritis & Rheumatism (1996) Vol. 39, No. 9 (supplement), S280); Naproxen (non-steroidal anti-inflammatory drug; see e.g., Neuro Report (1996) Vol. 7, pp. 1209-1213); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine (see e.g., Arthritis & Rheumatism (1996) Vol. 39, No. 9 (supplement), S281); Azathioprine (see e.g., Arthritis & Rheumatism (1996) Vol. 39, No. 9 (supplement), S281); ICE inhibitor (inhibitor of the enzyme interleukin-1β converting enzyme); zap-70 and/or lck inhibitor (inhibitor of the tyrosine kinase zap-70 or lck); VEGF inhibitor and/or VEGF-R inhibitor (inhibitors of vascular endothelial cell growth factor or vascular endothelial cell growth factor receptor; inhibitors of angiogenesis); corticosteroid anti-inflammatory drugs (e.g., SB203580); TNF-convertase inhibitors; anti-IL-12 antibodies; anti-IL-18 antibodies; interleukin-11 (see e.g., Arthritis & Rheumatism (1996) Vol. 39, No. 9 (supplement), S296); interleukin-13 (see e.g., Arthritis & Rheumatism (1996) Vol. 39, No. 9 (supplement), S308); interleukin-17 inhibitors (see e.g., Arthritis & Rheumatism (1996) Vol. 39, No. 9 (supplement), S120); gold; penicillamine; chloroquine; chlorambucil; hydroxychloroquine; cyclosporine; cyclophosphamide; total lymphoid irradiation; anti-thymocyte globulin; anti-CD4 antibodies; CD5-toxins; orally-administered peptides and collagen; lobenzarit disodium; Cytokine Regulating Agents (CRAs) HP228 and HP466 (Houghten Pharmaceuticals, Inc.); ICAM-1 antisense phosphorothioate oligo-deoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids; see e.g., DeLuca et al. (1995) Rheum. Dis. Clin. North Am. 21:759-777); auranofin; phenylbutazone; meclofenamic acid; flufenamic acid; intravenous immune globulin; zileuton; azaribine; mycophenolic acid (RS-61443); tacrolimus (FK-506); sirolimus (rapamycin); amiprilose (therafectin); cladribine (2-chlorodeoxyadenosine); methotrexate; antivirals; and immune modulating agents.

In one embodiment, the TNFα antibody, or antigen-binding portion thereof, is administered in combination with one of the following agents for the treatment of rheumatoid arthritis: small molecule inhibitor of KDR (ABT-123), small molecule inhibitor of Tie-2; methotrexate; prednisone; celecoxib; folic acid; hydroxychloroquine sulfate; rofecoxib; etanercept; infliximab; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1 TRAP; MRA; CTLA4-IG; IL-18 BP; ABT-874; ABT-325 (anti-IL 18); anti-IL 15; BIRB-796; SCIO-469; VX-702; AMG-548; VX-740; Roflumilast; IC-485; CDC-801; and mesopram. In another embodiment, an TNFα antibody, or antigen-binding portion thereof, is administered for the treatment of an TNFα-related disorder in combination with one of the above mentioned agents for the treatment of rheumatoid arthritis.

Non-limiting examples of therapeutic agents for inflammatory bowel disease with which an antibody, or antibody portion, of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-15, IL-16, IL-17, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90 or their ligands. The antibodies of the invention, or antigen binding portions thereof, may also be combined with agents, such as methotrexate, cyclosporin, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TNFα converting enzyme inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ).

Preferred examples of therapeutic agents for Crohn's disease in which an antibody or an antigen binding portion can be combined include the following: TNF antagonists, for example, anti-TNF antibodies, D2E7 (PCT Publication No. WO 97/29131; HUMIRA), CA2 (REMICADE), CDP 571, TNFR-Ig constructs, (p75TNFRIgG (ENBREL) and p55TNFRIgG (LENERCEPT)) inhibitors and PDE4 inhibitors. Antibodies of the invention, or antigen binding portions thereof, can be combined with corticosteroids, for example, budenoside and dexamethasone. Antibodies of the invention or antigen binding portions thereof, may also be combined with agents such as sulfasalazine, 5-aminosalicylic acid and olsalazine, and agents which interfere with synthesis or action of proinflammatory cytokines such as IL-1, for example, IL-1β converting enzyme inhibitors and IL-1ra. Antibodies of the invention or antigen binding portion thereof may also be used with T cell signaling inhibitors, for example, tyrosine kinase inhibitors 6-mercaptopurines. Antibodies of the invention, or antigen binding portions thereof, can be combined with IL-11. Antibodies of the invention, or antigen binding portions thereof, can be combined with mesalamine, prednisone, azathioprine, mercaptopurine, infliximab, methylprednisolone sodium succinate, diphenoxylate/atrop sulfate, loperamide hydrochloride, methotrexate, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, celecoxib, polycarbophil, propoxyphene napsylate, hydrocortisone, multivitamins, balsalazide disodium, codeine phosphate/apap, colesevelam hcl, cyanocobalamin, folic acid, levofloxacin, methylprednisolone, natalizumab and interferon-gamma

Non-limiting examples of therapeutic agents for multiple sclerosis with which an antibody, or antibody portion, of the invention can be combined include the following: corticosteroids; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α2b (Enzon/Schering-Plough), Copolymer 1 (Cop-1; COPAXONE; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-23, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD19, CD20, CD25, CD28, CD30, CD40, CD45, CD69, CD80, CD86, CD90 or their ligands. The antibodies of the invention, or antigen binding portions thereof, may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which the antibody or antigen binding portion thereof can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

The antibodies, or antigen binding portions thereof, used in the invention may also be combined with agents, such as alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists, IL-4 agonists.

Non-limiting examples of therapeutic agents for Angina with which an antibody, or antibody portion, can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil hcl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril, bisoprolol fumarate.

Non-limiting examples of therapeutic agents for Ankylosing Spondylitis with which an antibody, or antibody portion, can be combined in the methods and compositions of the invention include the following: ibuprofen, diclofenac and misoprostol, naproxen, meloxicam, indomethacin, diclofenac, celecoxib, rofecoxib, Sulfasalazine, Methotrexate, azathioprine, minocyclin, prednisone, etanercept, infliximab.

Non-limiting examples of therapeutic agents for Asthma with which an antibody, or antibody portion, in the methods and compositions of the invention can be combined include the following: albuterol, salmeterol/fluticasone, montelukast sodium, fluticasone propionate, budesonide, prednisone, salmeterol xinafoate, levalbuterol hcl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin hcl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, metaproterenol sulfate.

Non-limiting examples of therapeutic agents for COPD with which an antibody, or antibody portion, in the methods and compositions of the invention can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol hcl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast, Roflumilast.

Non-limiting examples of therapeutic agents for HCV with which an antibody, or antibody portion, in the methods and compositions of the invention can be combined include the following: Interferon-alpha-2a, Interferon-alpha-2b, Interferon-alpha con1, Interferon-alpha-n1, Pegylated interferon-alpha-2a, Pegylated interferon-alpha-2b, ribavirin, Peginterferon alfa-2b+ribavirin, Ursodeoxycholic Acid, Glycyrrhizic Acid, Thymalfasin, Maxamine, VX-497 and any compounds that are used to treat HCV through intervention with the following targets:HCV polymerase, HCV protease, HCV helicase, HCV IRES (internal ribosome entry site).

Non-limiting examples of therapeutic agents for Idiopathic Pulmonary Fibrosis with which an antibody, or antibody portion, in the methods and compositions of the invention can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone hcl, potassium chloride, triamcinolone acetonide, tacrolimus anhydrous, calcium, interferon-alpha, methotrexate, mycophenolate mofetil, Interferon-gamma-1β.

Non-limiting examples of therapeutic agents for Myocardial Infarction with which an antibody, or antibody portion, used in the methods and compositions of the invention can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hcl/mag carb, bumetanide, alteplase, enalaprilat, amiodarone hydrochloride, tirofiban hcl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine hcl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, cariporide.

Non-limiting examples of therapeutic agents for Psoriasis with which an antibody, or antibody portion, used in the methods and compositions of the invention can be combined include the following: small molecule inhibitor of KDR (ABT-123), small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hclbismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine.

Non-limiting examples of therapeutic agents for Psoriatic Arthritis with which an antibody, or antibody portion, used in the methods and compositions of the invention can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumab.

Non-limiting examples of therapeutic agents for Restenosis with which an antibody, or antibody portion, used in the methods and compositions of the invention can be combined include the following: sirolimus, paclitaxel, everolimus, tacrolimus, ABT-578, acetaminophen.

Non-limiting examples of therapeutic agents for Sciatica with which an antibody, or antibody portion, used in the methods and compositions of the invention can be combined include the following: hydrocodone bitartrate/apap, rofecoxib, cyclobenzaprine hcl, methylprednisolone, naproxen, ibuprofen, oxycodone hcl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone hcl, tizanidine hcl, diclofenac sodium/misoprostol, propoxyphene napsylate/apap, asa/oxycod/oxycodone ter, ibuprofen/hydrocodone bit, tramadol hcl, etodolac, propoxyphene hcl, amitriptyline hcl, carisoprodol/codeine phos/asa, morphine sulfate, multivitamins, naproxen sodium, orphenadrine citrate, temazepam.

Preferred examples of therapeutic agents for SLE (Lupus) in which an antibody or an antigen binding portion used in the methods and compositions can be combined include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. Antibodies of the invention or antigen binding portions thereof, may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-1ra. Antibodies of the invention or antigen binding portion thereof may also be used with T cell signaling inhibitors, for example, tyrosine kinase inhibitors; or molecules that target T cell activation molecules, for example, CTLA-4-IgG or anti-B7 family antibodies, anti-PD-1 family antibodies. Antibodies of the invention, or antigen binding portions thereof, can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. Antibodies of the invention or antigen binding portion thereof may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (PCT Publication No. WO 97/29131; HUMIRA), CA2 (REMICADE), CDP 571, TNFR-Ig constructs, (p75TNFRIgG (ENBREL) and p55TNFRIgG (LENERCEPT)).

Any one of the above-mentioned therapeutic agents, alone or in combination therewith, can be administered to a subject in combination with a TNF [symbol] antibody, or antigen-binding portion thereof, which is administered via pulmonary administration. The additional agent may also be administered through any means known to one of skill in the art, including, but not limited to, intraperitoneal, including intravenous or subcutaneous, oral, and pulmonary.

This invention is further illustrated by the following examples, which should not be construed as limiting. The contents of all references, patents and published patent applications cited throughout this application are incorporated herein by reference.

### EXAMPLE 1

### Systemic Delivery of a TNFα Inhibitor Via Pulmonary Delivery

### Inhalation Pharmacokinetics of Adalimumab (HUMIRA®) in Cynomolgus Monkeys

The following study describes the feasibility of adalimumab to achieve therapeutically desired systemic levels via inhalation using cynomolgus monkeys. One of the main goals of the study was to determine the pharmacokinetics of adalimumab administered via pulmonary means. This included administering nebulized aerosols of adalimumab to the lungs of anesthetized, tracheally intubated and ventilated monkeys at 10 mg/kg in two different modes of inhalation, followed by serum assay characterization of the inhalation pharmacokinetics of adalimumab. In addition, lung-regional distribution via each mode of inhalation was determined using a marker aerosol of fluorophore-labeled, non-absorbable dextran (FD-150S) administered in an identical fashion, followed by its direct recovery from different lung regions.

### Summary

The study used non-human primates of cynomolgus monkeys and determined the serum adalimumab concentration profiles and pharmacokinetics following two different (shallow and deep) modes of inhalation that targeted the central and peripheral airways, respectively. Such a targeting was accomplished by appropriate selection of inspiratory maneuvers in ventilation alongside tracheal intubation depth and nebulized aerosol size. The target lung-deposited dose was 10 mg/kg adalimumab, and its serum concentrations were measured for 16 days. Lung-regional distribution following each mode of inhalation was determined by a marker aerosol of fluoroscein isothiosyanate (FITC)-labeled dextran (FD-150S) administered in an identical fashion, followed by direct measurement of lung deposition in each of the dissected regions.

Nebulization of adalimumab appeared to be robust without causing appreciable degradation. All animals tolerated the aerosols, showing no signs of local or systemic distress, complications or abnormalities. In all animals, adalimumab clearly reached the systemic circulation following inhalation, yielding Cₘₐₓ of 2.31-5.91 mg/l over Tₘₐₓ of 2-4 days, followed by a mean systemic half-life (T_{1/2}) of 13.3±6.7 days. Kinetically, the lung absorption of adalimumab was not rate-determining; the terminal half-lives reflected elimination from the systemic circulation. Note, however, that the profiles displayed sudden drops in antibody levels at 10-12 days, possibly due to anti-human responses. Pharmacokinetic analysis yielded values of 0.99-4.18% for absolute bioavailability (F %) following these inhalations. Nevertheless, despite the successful targeting to the central (C) and peripheral (P) lung regions, represented by P/C ratios of 0.31 and 1.35, respectively, a difference in the F % values between the modes of inhalation was not significant. It is likely therefore that the upper bronchial delivery yielded lung absorption of adalimumab as much as the deep lung delivery, presumably by virtue of FcRn-mediated mechanisms.

In conclusion, adalimumab serum concentrations reached the therapeutically desired levels in humans, i.e., 5 mg/l, with a relatively faster Tₘₐₓ of 2-4 days, compared to subcutaneous injection.

### I. MATERIALS AND METHODS

Generally, animals were orotracheally intubated and ventilated in a Bird Mark 7A respirator circuit under anesthesia. HUMIRA® (50 mg/ml adalimumab) was nebulized in the circuit as 4.6 and 2.1 μm solution aerosols by in-line Aeronebs and administered to the lung at a target lung-deposited dose of 10 mg/kg in shallow and deep respiratory maneuvers, respectively. Then, serum concentrations of adalimumab were determined by a validated ELISA for 16 days to characterize inhalation pharmacokinetics, compared to intravenous injection profiles. Lung-regional distribution of these two modes of inhalation was separately determined with marker aerosols of FITC-labeled dextran (FD 150) by direct measurement of its lung deposition in the central and peripheral lung regions.

### I.A Materials

Adalimumab (D2E7; Humira®; 50 mg/ml) and a reference standard were used in the study. Each vial contained 40 mg of adalimumab in 0.8 mL of buffer solution and was used directly without dilution. Note that 2-4 vials were combined to prepare the dosing solution for nebulizers, in order to accomplish 10 mg/kg of the lung-deposited dose to each monkey, as described below. The antibody in non-biological and biological samples was determined be validated methods of ion exchange HPLC (IE-HPLC) coupled with UV detection at 280 nm and specific and sensitive ELISA, respectively.

Fluorescein isothiocyanate (FITC)-labeled dextran (FD-150S; weight averaged=150 kD) was purchased from Sigma-Aldrich (St. Louis, Mo.) and was used as a non-absorbable marker to determine lung-regional distributions following 2 different modes of inhalation used in this study. Its dosing solution was prepared at 20 mg/ml in 5 mM sterile phosphate-buffered saline (PBS; pH 7.4) and analysis employed validated gel permeation chromatography (GPC) coupled with fluorescence detection (excitation and emission wavelengths of 490 and 520 nm, respectively). The method was fully validated for both non-biological and biological samples, with respect to precision and accuracy <5% and linear response range of 7-600 ng/ml. Chemical agents use to prepare buffer solutions, such as PBS and IE-HPLC mobile phases were purchased from Fisher Scientific (Pittsburgh, Pa.) with the highest analytical grade.

### I.B Animals

A total of seven male cynomolgus monkeys (body weights of 2.6-3.0 kg at receipt) were used in the study. Each monkey was housed individually in a room which was tightly controlled for temperature, humidity, and dark-light cycling periods. Monkeys were carefully maintained and acclimatized according to the approved daily enrichment plan carried out by veterinarians and veterinary technicians. The animals were trained to extend either a forearm or a leg out the caging system, so that blood sampling became feasible under conscious condition in the pharmacokinetic study. There were no food or water restrictions.

Among seven monkeys, one animal was dedicated to several pilot experiments where two different modes of inhalation employed in this study, namely shallow and deep inhalation (INH-S and INH-D, respectively), were tested and optimized. Meanwhile, the remainder of six animals was divided into three and two groups during two phase studies of adalimumab pharmacokinetics following inhalation or intravenous injection and lung-regional distribution determination using FD-150S, respectively. Such a group assignment in each phase is described in Table 1.

TABLE 1
Experimental Group Assignments of 6 Monkeys

| | Pharmacokinetics | | Lung-regional distribution | |
|---|---|---|---|---|
| Animal | Route | Drug | Route | Drug |
| 1 | INH-S | adalimumab | INH-S | FD-150S |
| 2 | INH-S | adalimumab | INH-S | FD-150S |
| 3 | INH-D | adalimumab | INH-D | FD-150S |
| 4 | INH-D | adalimumab | INH-D | FD-150S |
| 5 | IV | adalimumab | INH-S | FD-150S |
| 6 | IV | adalimumab | INH-D | FD-150S |

INH-S, shallow inhalation; INH-D, deep inhalation; IV, intravenous injection; FD-150S, flourescein isothiocyanate (FITC)-labeled dextran (MW: 150 kDa) Target dose: 10 mg/kg for adalimumab and 2.5 mg/kg for FD-150S

### I.C Adalimumab Pharmacokinetics in Monkeys: Inhalation Pharmacokinetics

Animals (body weights of 3.0-3.8 kg; n=2 for INH-S and INH-d; Table 1) were anesthetized with a combination of intramuscular injections of atropine sulfate at 0.1 mg/kg (0.4 mg/ml; American Regent), ketamine hydrochloride at 10.0 mg/kg (Ketaject®; 100 mg/ml; Phoenix Pharmaceuticals) and xylazine at 1.0 mg/kg (Xyla-Ject®; 20 mg/ml; Phoenix Pharmaceuticals) for adalimumab delivery to the lung via inhalation. Under stable anesthesia, each of the animals was orotracheally intubated with a cuffed endotracheal (ET) tube (3.0 mm I.D. and 4.2 mm O.D.; Hudson Respiratory Care) and ventilated with a pneumatically powered Bird Mark 7A respirator (VIASYS Healthcare). Their vital signs, e.g., heart rate, blood pressure, body temperature and % of oxygen saturation (SpO₂), as well as eye blink response to palpebral stimulation were monitored about every 10 minutes throughout the procedure to ensure the adequacy of anesthesia and the absence of abnormality. Two types of Aeroneb Lab micropump nebulizers (Aerogen, Galway, Ireland) that generated different sizes adalimumab aerosols were used in-line with the Mark 7A respirator circuit. Hence, by differently employing the following choice of the tracheal intubation depth, respirator set up and aerosol size, as shown in Table 2, shallow and deep lung-regional distributions could be targeted, while maintaining a comparable lung dose of 10 mg/kg.

Because preliminary studies predicted 30-40% of adalimumab loaded in the nebulizers to be deposited into the lung in this system, 1.5-2.8 ml of 50 mg/ml adalimumab solution (equivalent to 75-140 mg) was filled in the nebulizer and aerosolized under each respirator setup shown in Table 2, so that target lung-deposited dose of 10 mg/kg could be accomplished. During the nebulization, non-deposited, exhaled adalimumab aerosols were collected at the exit of the respirator circuit using a disposable in-line filter (Sterivent®; Tyce Healthcare). While the nebulization was terminated at the dryness of the nebulizer cup by 6-10 minutes, the ventilation continued for further 5 minutes; the ET tube was removed, when the animals regained consciousness from the anesthesia, typically at 1 hour following anesthetic induction.

TABLE 2
Experimental setups to target shallow
and deep lung-regional distribution

Targeted lung-regional distribution

| | Shallow (INH-S) | Deep (INH-D) |
|---|---|---|
| Intubation depth | 12 cm | 13.5 cm |

Respirator setup

| | | |
|---|---|---|
| Pressure: | 13-15 cm H₂O | 32 cm H₂O |
| Breath cycle: | 27-30 breaths/min | 20 breaths/min |
| Breath hold: | 0.3 sec | 3 sec |
| Aerosol size¹ | 4.6 μm | 2.1 μm |

¹Mass median aerodynamic diameter (MMAD) determined for adalimumab aerosolized into the respirator circuit at a continuous air-flow and collected in the Next Generation Pharmaceutical Impactor at a vacuum flow rate of 15 l/min.

At the completion of administration, adalimumab remaining in the nebulizer, respirator circuit and exhalation filter was recovered with 100 ml of PBS and determined by the IE-HPLC method. Hence, the “actual” lung deposited dose of adalimumab in each experiment was estimated via its subtraction from the loaded mass in the nebulizer (50 mg/ml multiplied by 1.5-2.8 ml of loaded volume). Following inhalation, venous blood samples (1.2 ml) were withdrawn at different time intervals of 0.5, 1, 2, 3, 6, 12, and 24 hours followed by 2, 4, 6, 8, 10, 12, 14, and 16 day. Note that this blood sampling was undertaken from the conscious animals after recovery of anesthesia typically at 1 hour post-inhalation. Serum samples were obtained via centrifuge at 2,800 g for 10 minutes at 24 degrees C. and the stored at −70 degrees C. prior to analysis for adalimumab determination by ELISA. The serum assay was set blind with respect to the group assignments. The animals were carefully monitored daily during and following each study with respect to any indications of respiratory complications related to intubation or adalimumab exposure. This included observations for normality in mucosal color respiration, behavior and appetite and/or the absence of cough or dyspnea.

### I.D Adalimumab Pharmacokinetics in Monkeys: Intravenous Pharmacokinetics

Animals (body weights of 4.1 and 3.6 kg; n=2 for IV; Table 1) were anesthetized and orotracheally intubated in an identical fashion to the studies of inhalation pharmacokinetics described above. Following observations of adequate anesthesia under normal vital signs, 0.82 and 0.72 ml of 50 mg/ml adalimumab solution, respectively, was injected intravenously through lateral saphenous vein within 3 minutes, accomplishing a dose of 10 mg/kg. Venous blood samples (1.2 ml) were withdrawn following injection at different time intervals of 0.5, 1, 2, 4, 6, 12, and 24 hours, followed by 2, 4, 6, 8, 10, 12, 14, and 16 day, and their serum stored at −70 degrees C. prior to analysis. The sampling and storage procedures used were the same as those described above. Daily animal monitoring was similarly carried out to ensure the absence of abnormality.

### I.E Data Analysis

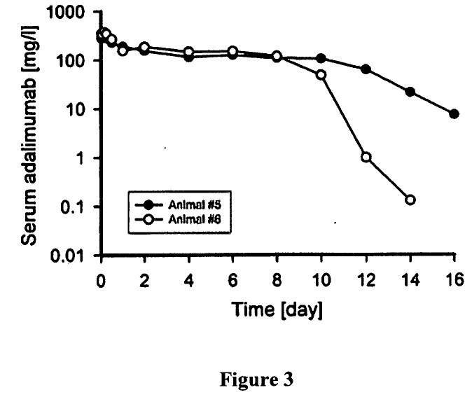

Each of the serum adalimumab profiles shown in FIGS. 2 and 3 represents each individual animal, enabling calculation of the following pharmacokinetic parameters: the maximum serum concentration (Cₘₐₓ) and the time to reach Cₘₐₛ (Tₘₐₓ) by data observation, the terminal phase rate constant (λ) and half-life (T_{1/2}=0.693/λ) by the natural log-linear regression of the profiles between 4 and 8 days, the area under the serum concentration vs. time curve by 8 days (AUC₀₋₈ day) by the trapezoidal method and those by time infinity (AUC_{0-∞}) from AUC₀₋₈ day plus extrapolated AUC, the total body systemic clearance (CL) from dose/AUC_{0-∞}, and the apparent volume of distribution at steady-state (V_{SS}) from CL multiplied by mean residence time (MRT), the latter being calculated from the moment analysis from the serum adalimumab profiles.

It should be noted that there was a compromise in some of these parameter estimations due to an extremely long systemic half-life (14 days) of adalimumab and unexpected drops in its serum concentrations following 10 day (FIGS. 2 and 3), as discussed below. Hence, the absolute bioavailability (F %) of adalimumab following inhalation was calculated in two ways, denoted as F %₀₋₈ day and F %_{0-∞}, using dose-normalized AUC₀₋₈ day and AUC_{0-∞}, respectively.

### I.F. Lung-Regional Distribution Following Inhalation in Monkeys

The experiment was carried out following sufficient washout period of about 30 days in order to minimize possible interactions and/or complications. Animals (body weights 3.2-4.3 kg; n=3 for INH-S and INH-D; Table 1) were anesthetized, orotracheally intubated and ventilated in an identical fashion to the studies of inhalation pharmacokinetics described above, including two different (shallow and deep) modes of inhalation (Table 2). Following observations of adequate anesthesia under normal vital signs, 2.0 ml of 20 mg/ml FD-150S solution (PBS; pH 7.4) loaded in the nebulizers was aerosolized in the respirator circuit until dryness for 6-8 minutes and thereby, administered to the animals at a target dose of 2.5 mg/kg under the shallow and deep inspiratory maneuvers described in Table 2. Non-deposited, exhaled FD-150S was collected by the filter at the exit of the respirator circuit. At the completion of administration, FD-150S remaining in the nebulizer, respirator circuit and exhalation filter was recovered with 250 ml of PBS and then, determined by the validated fluorescence-GPC. Hence, the “actual” lung-deposited dose of FD-150S was estimated in each experiment via its subtraction from the initial loaded FD-150S filled in the nebulizer cup (40 mg; 20 mg/ml multiplied by 2.0 ml).

Immediately after administration, while still under anesthesia by ketamine/zylazine/atropine, each of the animals was euthanized with an intravenous injection of Euthasole (390 mg/ml pentobarbital sodium and 50 mg/ml phenyloin sodium, Virbac AH) at a dose of 0.5 ml/kg. Their chest cavity was opened and then, the lung lobes, trachea, and bronchus were surgically removed en bloc; these were frozen at −70 degrees C. prior to analysis. Determination of lung-regional distribution was commenced by regional tissue dissection into trachea, bronchus, and inner and outer, i.e., central and peripheral, regions of each lung lobe, as described in FIG. 1; each lung lobe was dissected in half in weight, named as the central and peripheral regions. Each dissected tissue was homogenized in 10 volumes PBS with a biohomogenizer (Biospec Products) and centrifuged at 2,800 g for 15 minutes at 10 degrees C. Following appropriate dilution, the supernatants were filtered with 0.2 μm syringe filters (15 mm; regenerated cellulose; Corning) and analyzed with respect to FD-150S by the validated fluorescence-GPC. FD-150S recovered from each of the inner and outer regions of 7 lung lobes (FIG. 1) were combined to yield the central and peripheral lung lobar depositions, respectively. Then, a peripheral-to-central (P/C) distribution ratio for each mode of inhalation was calculated from the peripheral lung-deposited mass divided by a sum of FD-150S recovered from the central lung, trachea, and bronchus.

### II. RESULTS

Collections of adalimumab nebulized into the respirator circuit by the Next Generation Pharmaceutical Impactor for aerosol size characterization verified the robustness of the antibody without causing appreciable degradation, evidenced by their IE-HPLC chromatograms unaltered from those for the reference standard. All animals well tolerated the aerosols, exhibiting no signs of local or systemic adverse effects, regardless of the treatments. All vital signs monitored during the anesthetic periods for administration were stable within normal ranges with anesthesia, e.g., 90-125 bpm in heart rate, 115-180 mmHg in blood pressure, 36.0-37.6 degrees C. in body temperature and 83-99% in SpO₂. There were no signs of distress, complications or abnormality throughout. Visual examination of the lung of each animal at its removal in the studies of lung-regional distribution concluded normal appearance by the absence of edema or mucosal color change.

Generally, all animals tolerated the adalimumab aerosols with no signs of local or systemic distress, complications or abnormality. Lung-deposited doses of adalimumab were 10.3-14.0 mg/kg, yielding Cₘₐₓ of 2.3-5.9 mg/l at Tₘₐₓ of 2-4 days, followed by a mean systemic half-life of 13.3±6.7 days. Its lung absorption was not rate-determining kinetically, showing the terminal half-life consistent with that following intravenous injection. However, the serum profiles displayed a sudden drop in the antibody level 10-12 days post-inhalation and -injection, possibly due to anti-human responses. Hence, pharmacokinetic analysis was carried out with the serum data for 10 days, yielding the absolute bioavailability (F %) of 1.0-4.2% following inhalation. Notably, a difference in any pharmacokinetic parameters was not significant between two modes of inhalation, despite their successful targeting to the central (C) and peripheral (P) lung regions, represented by the FD150's P/C ratios of 0.31 and 1.35, respectively.

### II.A Adalimumab Pharmacokinetics in Monkeys

Serum adalimumab concentration vs. time profiles following 2 different modes of inhalation and the derived pharmacokinetic parameters are shown in FIG. 2 and Table 3, respectively. The estimated lung-deposited doses were in a range of 10.3-14.0 mg/kg (Table 3), successfully being consistent with the target dose of 10 mg/kg. In all animals, the antibody clearly reached the systemic circulation, while Cₘₐₓ ranging 2.31-5.91 mg/l (3.88±1.57 mg/l; Table 3) remained slightly lower than, or comparable to, the desired antibody levels in humans, i.e., 5 mg/l (prescription information on adalimumab package insert). Notably, T was found late, appearing over 2-4 days, which implied rather slow absorption of adalimumab from the lung, regardless of inhalation mode. In contrast, similar studies for inhaled Fc-fusion proteins of erythropoietin (Epo) and follicle-stimulating hormone (FSH) in monkeys reported a shorter ≦2 days of Tₘₐₓ (Bitonti et al. (2004); Low et al. (2005) Hum Reprod 20:1805-1813).

Interestingly, the profiles consistently displayed sudden drops in serum concentrations at 10-12 days, resulting in negligible antibody levels below the limit of quantitation after 12 days of inhalation (FIG. 3). Such a drop may be the result of a possible anti-human immune response to adalimumab developed in the monkeys. Indeed, similar observations clearly continued in the animals receiving an intravenous injection, as shown in FIG. 3. For this reason, subsequent pharmacokinetic analysis, such as λ, T_{1/2}, AUC, and F % determination, employed serum data obtained by 8 days only, despite possible compromise in the profile extrapolation. Even so, the averaged T_{1/2} in four monkeys from the data for 4-8 days was 13.3±6.7 days (6.8-20.4 days; Table 3), the value being equivalent to that in humans (14 days) (Humira prescription information; package insert).

The serum concentration profiles of adalimumab following intravenous injection at 10 mg/kg are shown in FIG. 3, yielding the pharmacokinetic parameters tabulated in Table 4. Although the profiles appeared to be bi-phasic by 10 days, similar unexpected drops in antibody levels were observed thereafter. Nevertheless, T_{1/2} obtained from the serum data by 8 days were 14.7 and 10.8 days (Table 4), the values insignificantly differing from those seen in the inhalation profiles (Table 3). It is likely therefore that adalimumab absorption from the lung was not kinetically rate-limiting, and rather, its elimination from the systemic circulation was the slowest kinetically. Notably, the intravenous profiles shown in FIG. 3 derived a low CL of 0.12 and 0.13 ml/hr/kg with a small V_{SS} of 18.9 and 19.4 ml/kg; the corresponding values in humans have been reported to be 0.17 ml/hr/kg and 67-68 ml/kg (Humira prescription information; package insert).

Because the serum adalimumab concentrations unexpectedly dropped to the negligible levels after 10 days (see FIGS. 2 and 3), options to further analyze such profiles were limited to either the use of the data by 8 days without considering post-8 day contribution or the inclusion of kinetic extrapolation to yield the data after 8 days by time infinity. Correspondingly, the values for AUC and F % determined based on these 2 options are shown in Tables 3 and 4. Note that F % calculation for inhalation also took dose-normalization in each animal into account, assuming adalimumab's linear pharmacokinetics in monkeys. As a result, regardless of the data treatments, F % was estimated to range from 0.99 to 4.18% (Table 3).

### II.B Effects of Shallow and Deep Lung-Regional Distribution

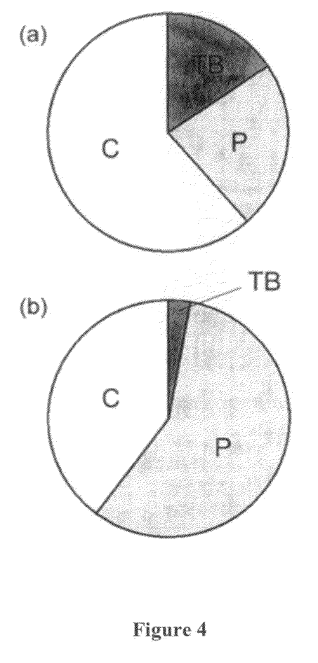

Lung-regional distributions and FD-50S following two different modes of inhalation employed in this study are shown in Table 5 and FIG. 4. The lung-deposited doses in 6 monkeys ranged from 2.18-3.82 mg/kg (2.90±0.72 mg/kg; Table 5), reasonably consistent with the target dose of 2.5 mg/kg. Moreover, the analysis yielded ≧90% (93.7±3.3%; Table 5) of recovery over the lung-deposited dose, suggesting that our method of lung-deposited dose estimation was valid and resultant lung-regional distribution data likely represented the actual distribution for adalimumab. As a result, the shallow and deep inspiratory maneuvers alongside manipulations in intubation depth and aerosol size (Table 2) indeed caused major (˜60%) central and peripheral lung-regional distributions, respectively, as shown in Table 5 and FIG. 4.

Table 6 summarized the averaged bioavailability (F %) of adalimumab following each of the shallow and deep inhalation, as to the P/C ratios of the lung-regional distribution calculated from the data shown in Table 5. The difference in these F % values between the modes of inhalation was not convincing without clear distinction, despite successful central and peripheral lung delivery with the P/C ratios if 0.31 and 1.35, respectively; in both cases, the averaged F % from two animals in each group ranged from 1.7-2.6%. It is likely, therefore, that the upper bronchial airway delivery yielded lung absorption of adalimumab as much as the deep lung delivery, presumably due to FcRn-mediated mechanisms.

TABLE 3
Pharmacokinetic parameters derived from the serum adalimumab concentration vs. time profiles by 8 hours following
2 modes of inhalation at a nominal dose of 10 mg/kg in 4 monkeys. The profiles are shown in FIG. 2.

| | | | LDD | Cₘₐₓ | Tₘₐₓ | T_{1/2} | AUC_{0-8 hr} | AUC_{0-∞} | F %_{0-8 hr} | F %_{0-∞} |
|---|---|---|---|---|---|---|---|---|---|---|
| Animal | Code | Inhalation | [mg/kg] | [mg/l] | [day] | [day] | [mg/l-day] | [mg/l-day] | [%] | [%] |
| #1 | AB05-M3 | Shallow | 14.0 | 5.91 | 4 | 20.4 | 41.69 | 193.25 | 2.41 | 4.18 |
| #2 | AB05-M5 | Shallow | 12.3 | 3.06 | 4 | 6.8 | 19.96 | 40.03 | 1.31 | 0.99 |
| #3 | AB05-M4 | Deep | 10.3 | 2.31 | 2 | 17.7 | 16.48 | 61.85 | 1.30 | 1.83 |
| #4 | AB05-M6 | Deep | 11.6 | 4.24 | 4 | 8.4 | 30.57 | 65.71 | 2.13 | 1.77 |
| Average ± SD | | | 12.1 ± 1.5 | 3.88 ± 1.57 | 3.5 ± 1.0 | 13.3 ± 6.7 | | | 1.78 ± 0.57 | 2.19 ± 1.38 |

LDD, lung-deposited dose; Cₘₐₓ, the observed maximum serum concentration; Tₘₐₓ, the observed time to reach Cₘₐₓ; T_{1/2}, the systemic half-life; AUC_{0-8 hr}, the area under the serum concentration vs. time (AUC) by 8 hours; AUC_{0-∞}, AUC by time infinity; F %_{0-8 hr}, the absolute bioavailability calculated from AUC_{0-8 hr}; F %_{0-∞}, the absolute bioavailability calculated from AUC_{0-∞}

TABLE 4
Pharmacokinetic parameters derived from the serum adalimumab concentration
vs. time profiles by 8 hours following intravenous injection at a dose
of 10 mg/kg in 2 monkeys. The profiles are shown in FIG. 3.

| | | Dose | T_{1/2} | CL | Vₛₛ | AUC_{0-8 hr} | AUC_{0-∞} |
|---|---|---|---|---|---|---|---|
| Animal | Code | [mg/kg] | [day] | [ml/hr/kg] | [ml/kg] | [mg/l-day] | [mg/l-day] |
| #5 | AB05-M1 | 10.0 | 14.7 | 0.12 | 18.9 | 1151.89 | 3430.86 |
| #6 | AB05-M2 | 10.0 | 10.8 | 0.13 | 19.4 | 1324.76 | 3169.40 |
| Average | | 10.0 | 12.8 | 0.13 | 19.2 | 1238.33 | 3300.13 |

T_{1/2}, the systemic half-life; CL, the systemic total body clearance; Vₛₛ, the apparent volume distribution at a steady-state; AUC_{0-8 hr}, the area under the serum concentration vs. time (AUC) by 8 hours; AUC_{0-∞}, AUC by time infinity.

TABLE 5
Lung-regional distributions of FD-150S following 2 modes of
inhalation at a nominal dose of 2.5 mg/kg in 6 monkeys.

| | | LDD | % Recovery | % Distribution | | |
|---|---|---|---|---|---|---|
| Animal | Inhalation | [mg/kg] | [%] | TB | C | P |
| #1, 2, 5 | Shallow | 2.26 ± 0.10 | 95.3 ± 4.1 | 15.72 ± 4.68 | 61.44 ± 9.68 | 22.83 ± 7.78 |
| #3, 4, 6 | Deep | 3.54 ± 0.25 | 92.2 ± 2.0 | 2.97 ± 1.34 | 39.72 ± 4.07 | 57.31 ± 3.17 |

LDD, lung-deposited dose; TB, tracheobronchi; C, central lung lobes; P, peripheral lung lobes
% Recovery = [total FD-150S recovered from a whole lung]/[lung-deposited dose] × 100
% Distribution = [FD-150S recovered from each of the lung-regional sections]/[total FD-150S recovered from a whole lung] × 100

To highlight an issue associated with previous finding of Epo-Fc fusion protein (Bitonti et al. 2004), the dose-normalized AUC_{0-∞} (AUC_{0-∞} divided by the lung-deposited dose) was calculated from the serum concentration profiles of adalimumab (FIG. 2) and Epo-Fc (Bitonti et al. (2004)) following shallow and deep inhalation in monkeys. These are summarized in Table 7. Notably, the dose-normalized AUC_{0-∞} of these Fc-molecules was similar (8.3 vs. 6.3 kg·day/l, respectively; Table 7) following shallow inhalation, suggesting their almost comparable absorption kinetics, presumably due to FcRn-mediated mechanisms. Thus, a difference in the lung absorption kinetics for these Fc-molecules rather existed in those from the peripheral lung region, displaying quite minute absorption for Epo-Fc, despite the lung membrane generally exhibiting favorable features for drug absorption. Obviously, this deduction assumed parallel systemic disposition kinetics between adalimumab and Epo-Fc, which was most likely the case by virtue of similar systemic half-lives (14 and 16 days, respectively) (Bitonti et al. (2004); Humira package insert).

TABLE 6
Averaged absolute bioavailability (F %) for adalimumab
following shallow and deep inhalation, as to their P/C
ratios of lung-regional distribution in monkeys.

| Inhalation | P/C ratio | F %_{0-8 hr} | F %_{0-∞} |
|---|---|---|---|
| Shallow | 0.31 | 1.86 | 2.59 |
| Deep | 1.35 | 1.72 | 1.80 |

P/C ratio, the peripheral-to-central lung distribution ratio; F %_{0-8 hr}, and F %_{0-∞}, the absolute bioavailability values calculated from AUC_{0-8 hr} and AUC_{0-∞}, respectively

TABLE 7
Dose-normalized AUC_{0-∞} calculated from the serum concentration
profiles of adalimumab (FIG. 2) and Epo-Fc[2] following
shallow and deep inhalation in monkeys.

| | | |
|---|---|---|
| MW | LDD | Dose-normalized AUC_{0-∞} |

| Molecule | [kDa] | [mg/kg] | Shallow | Deep |
|---|---|---|---|---|
| Adalimumab | 148 | 10.0 | 8.3 | 5.8 |
| Epo-Fc | 112 | 0.3 | 6.3 | 2.3 |

Dose-normalized AUC_{0-∞} = [AUC_{0-∞}]/[lung-deposited dose] (kg · day/l)

### III. CONCLUSION

Adalimumab inhalation at 10 mg/kg reached its therapeutically desired serum levels, i.e., 4 mg/l, with a relatively faster Tₘₐₓ of 2-4 days, despite a low F % of 1.0-4.2%. The upper bronchial airway delivery appeared to yield lung absorption of adalimumab as much as the deep lung delivery, presumably by virtue of FcRn-mediated mechanisms. The above study in monkeys demonstrated that, while lung delivery of adalimumab accomplished the systemic antibody levels (2.31-5.91 mg/l; Table 3), equivalent to those desired in the subcutaneous regimen in humans, at relatively faster Tₘₐₓ of 2-4 days, its absolute bioavailability (F %) remained low at 0.99-4.18%. This appears to be consistent with recent findings, including those described herein, which suggest that FcRn-mediated transcytotic absorption from the lung is a high affinity and low capacity system and thereby, its rate in rats remained ≦100 ng/hr (Kim et al. (2004) Am J Physiol 287:L616; Sakagami et al. (2006) Pharm Res 23:270; and Sakagami et al. (2006) Respiratory Drug Delivery X, 1:57). Furthermore, in the airways, alveolar macrophages were shown to phagocytose IgGs and Fc-molecules possibly including adalimumab, further reducing the depot for lung absorption and thereby, causing the low F % (Lonbry et al. (2004) Am J Physiol 286:L1002). Emerging evidence suggests TNFα is a promising therapeutic target for asthma and its inhibition has been shown to improve airway hyper-reactivity and lung function in patients (Russo et al. (2005) Clin Sci (Lond) 109:135; Howarth et al. (2005) Thorax 60:1012; Berry et al. (2005) Proc Am Throacic Sco 2:A569). In this case, adalimumab alone may be advantageous for the sake of maximized long durations of local action with lower systemic levels.

In sum, adalimumab was administered to the lungs of cynomolgus monkeys, and its serum pharmacokinetics were characterized following 2 different modes of inhalation targeting the central and peripheral airways. In all animals, adalimumab clearly reached the systemic circulation via inhalation, yielding Cₘₐₓ of 2.31-5.91 mg/l over 2-4 days, followed by a mean systemic T_{1/2} of 13.3±6.7 days. Kinetically, the lung absorption of adalimumab was not rate-determining; the terminal half-lives reflected elimination from the systemic circulation. Note, however, that the profiles displayed sudden drops in antibody levels at 10-12 days, possibly due to anti-human responses. Pharmacokinetic analysis yielded values of 0.99-4.18% for absolute bioavailability (F %) following these inhalations. Adalimumab was successfully targeted to the central (C) and peripheral (P) lung regions, represented by P/C ratios of 0.31 and 1.35, respectively. A difference in the F % values between the modes of inhalation was not significant. It is likely therefore that the upper bronchial delivery yielded lung absorption of adalimumab as much as deep lung delivery, presumably due to FcRn-mediated mechanisms. Even so, by virtue of 10 mg/kg of the lung-deposited dose, the serum concentrations reached the therapeutically desired levels in humans, i.e., 5 mg/l, with a relatively faster Tₘₐₓ of 2-4 days, compared to subcutaneous injection (Humira package insert).

### EXAMPLE 2

### Robustness of a Tnfα Inhibitor for Pulmonary Delivery

### L929 Antigen Neutralization Bioassay for Aerosolized Adalimumab (HUMIRA®)

The following study describes the robustness of adalimumab via nebulization for inhalation delivery using L929 Antigen neutralization bioassay. The study was made to ensure that the bioactivity of adalimumab is not compromised via nebulization.

An Aeroneb Lab nebulizer, generating 2.1 μm aerosols, was used in-line within the ventilation circuit with the Bird Mark 7A respirator for nebulization of 2.5 ml solution of adalimumab (HUMIRA®; 50 mg/ml). This setup was identical to that employed in Example 1, except that it was performed without animals. Under the shallow and deep inspiratory maneuvers controlled by the respirator (Table 2), the nebulized aerosols were recovered by the bubble trap with 50 mM phosphate-buffered saline (pH 7.4), and the samples were stored at −70° C. prior to the bioassay. L929, a murine fibrosarcoma cell line (#ATCC CCl 1 NCTC clone 929), was used as an antigen neutralization assay (Aggarwal, B. B. et al. (1985) J. Biol. Chem. 260, 2345-2354). Cells were cultured in RPMI and 10% fetal bovine serum. Various amounts of the diluted adalimumab control (i.e., not nebulized) or nebulized samples of adalimumab were mixed with 500 pg/ml antigen, human tumor necrosis factor α (hTNFα), and incubated at 37° C. for 30 minutes in 96-well plates. Subsequently, 50,000 cells were added to each well alongside actinomycin-D at 1 μg/ml as a metabolic inhibitor. Following incubation at 37° C. for 18 hours, the cells were lysed by addition of 501 of 20% SDS and overnight incubation at 37° C. Each lysate sample was subjected to optical density (OD) measurement at dual wavelengths of 570 and 630 nm with a 96 well plate reader. The IC₅₀ values of neutralizing antibodies were determined from a sigmoidal relationship of the OD values and adalimumab concentration using a nonlinear regression curve-fitting program, GraphPad Prism (GraphPad Software, Inc., La Jolla, Calif.). While the cell viability was ensured prior to the cell lysis by the MTT (3-{4,5-dimethylhiazol-2-yl} 2,5-diphenyltetrazolium bromide) method, low OD values ranging from 0.07 to 0.15 was indicative of cell death.

As in many bioassays of this type, a less than 2-fold difference in the IC₅₀ values was considered within the technical limitation of the assay, which thereby would conclude that the bioactivity of the test samples was not compromised. Table 8 shows the derived IC₅₀ values obtained from each of the test samples of adalimumab. Regardless of the shallow or deep inspiratory maneuvers (INH-S and INH-D, respectively), the bioactivity of adalimumab aerosolized by the Aeroneb Lab nebulizer was shown to change no more than 2-fold. This demonstrated the robustness of adalimumab via nebulization for inhalation delivery.

TABLE 8
IC₅₀ values for adalimumab subjected to nebulization,
derived from L929 Antigen neutralization bioassay

| Control | Shallow (INH-S) | Deep (INH-D) |
|---|---|---|
| 16.60 | 14.98 | 11.82 |
| 13.76 | 13.47 | 7.60 |
| 16.33 | | |

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The practice of the present invention will employ, unless otherwise indicated, conventional techniques of immunology, molecular biology and cell biology, which are well known in the art.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

What is claimed:

1. A method of treating a subject having a disorder in which TNFα activity is detrimental comprising pulmonary delivery of a TNFα inhibitor to the subject, such that the disorder in which TNFα is detrimental is treated.

2. A method of achieving systemic circulation of a TNFα inhibitor in a subject comprising administering the TNFα inhibitor to the central and peripheral lung region of the subject via inhalation, such that systemic circulation of the TNFα inhibitor is achieved.

3. A method of achieving systemic circulation of a TNFα inhibitor in a subject comprising administering the TNFα inhibitor to the peripheral lung region of the subject via inhalation, such that systemic circulation of the TNFα inhibitor is achieved.

4. The method of any one of claims 1-3, wherein the TNFα inhibitor is formulated in a composition suitable for inhalation.

5. The method of claim 4, wherein the composition is selected from the group consisting of an inhalable powder, a propellant-containing aerosol, and a propellant-free inhalable solution.

6. The method of claim 5, wherein the inhalable powder is administered to the subject via a dry powder inhaler (DPI).

7. The method of claim 5, wherein the propellant-containing aerosol is administered to the subject via a metered dose inhaler (MDI).

8. The method of claim 5, wherein the propellant-free inhalable solution is administered to the subject via a nebulizer.

9. The method of any one of claims 1-8, further comprising achieving a Tₘₐₓ of less than or equal to about 4 days for the TNFα inhibitor.

10. The method of any one of claims 1-8, wherein the TNFα inhibitor is distributed to the central lung region of the subject such that a P/C ratio of about 0.3 is achieved.

11. The method of any one of claims 1-8, wherein the TNFα inhibitor is distributed to the peripheral lung region of the subject such that a P/C ratio of about 1.3 is achieved.

12. The method of any one of claims 1-8, wherein a maximum serum concentration (Cₘₐₓ) of at least about 2.3 mg/L of the TNFα inhibitor is achieved.

13. The method of any one of claims 1-8, wherein a Cam of at least about 4.2 mg/L of the TNFα inhibitor is achieved.

14. The method of any one of claims 1-8, wherein a Cₘₐₓ of at least about 5 mg/L of the TNFα inhibitor is achieved.

15. The method of any one of claims 1-8, wherein at least one pharmacokinetic characteristic selected from the group consisting of a Tₘₐₓ of less than or equal to about 4 days, an absolute bioavailability (F %) of at least about 0.99%, and a Cₘₐₓ of at least about 2.3 mg/L, is achieved following administration of the TNFα inhibitor.

16. The method of claim 15, wherein a Tₘₐₓ of about 2 to about 4 days is achieved following administration of the TNFα inhibitor.

17. The method of claim 15, wherein a Cₘₐₓ of about 2.3 to about 5.9 mg/L is achieved following administration of the TNFα inhibitor.

18. The method of any one of claims 1-17, wherein the subject is a human.

19. The method of any one of claims 2-17, wherein the subject has a disorder in which TNFα activity is detrimental.

20. The method of claim 1 or 19, wherein the disorder in which TNFα activity is detrimental is selected from the group consisting of an autoimmune disorder, a spondyloarthropathy, an intestinal disorder, a skin disorder, and a pulmonary disorder.

21. The method of claim 20, wherein the autoimmune disorder is rheumatoid arthritis or juvenile rheumatoid arthritis.

22. The method of claim 20, wherein the spondyloarthropathy is ankylosing spondylitis or psoriatic arthritis.

23. The method of claim 20, wherein the intestinal disorder is Crohn's disease.

24. The method of claim 20, wherein skin disorder is psoriasis.

25. The method of claim 20, wherein pulmonary disorder is chronic obstructive pulmonary disease or asthma.

26. The method of any one of claims 1-25, wherein the TNFα inhibitor is a TNFα antibody, or antigen-binding portion thereof, or a fusion protein.

27. The method of claim 26, wherein the fusion protein is etanercept.

28. The method of claim 26, wherein the TNFα antibody, or antigen-binding portion thereof, is selected from the group consisting of infliximab, golimumab, and adalimumab.

29. The method of claim 26, wherein the TNFα antibody, or antigen-binding portion thereof, is an antibody selected from the group consisting of a humanized antibody, a chimeric antibody, a human antibody, and a multivalent antibody.

30. The method of claim 29, wherein the human TNFα antibody, or antigen-binding portion thereof, dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

31. The method of claim 29, wherein the human TNFα antibody, or antigen-binding portion thereof, has the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

32. The method of claim 29, wherein the human TNFα antibody, or antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

33. The method of claim 29, wherein the human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

34. A method of treating a pulmonary disorder in a subject comprising pulmonary delivery of a TNFα inhibitor to the subject, wherein the pulmonary administration comprises local delivery of the TNFα inhibitor to the lung(s) of the subject.

35. The method of claim 34, wherein the pulmonary disorder is asthma or chronic obstructive pulmonary disease (COPD).

36. The method of claim 34 or 35, wherein the TNFα inhibitor is a TNFα antibody, or antigen-binding portion thereof, or a fusion protein.

37. The method of claim 36, wherein the fusion protein is etanercept.

38. The method of claim 37, wherein the TNFα antibody, or antigen-binding portion thereof, is selected from the group consisting of infliximab, golimumab, and adalimumab.

39. The method of claim 36, wherein the TNFα antibody, or antigen-binding portion thereof, is an antibody selected from the group consisting of a humanized antibody, a chimeric antibody, a human antibody, and a multivalent antibody.

40. The method of claim 39, wherein the human TNFα antibody, or antigen-binding portion thereof, dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

41. The method of claim 39, wherein the human TNFα antibody, or antigen-binding portion thereof, has the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

42. The method of claim 39, wherein the human TNFα antibody, or antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

43. The method of claim 39, wherein the human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

44. A pharmaceutical composition comprising a TNFα antibody and a pharmaceutically acceptable carrier, wherein the pharmaceutical composition is suitable for inhalation by a subject and is selected from the group consisting of an inhalable powder or a dry powder composition, a propellant-containing aerosol, and a propellant-free inhalable solution or suspension.

45. The pharmaceutical composition of claim 44, wherein the pharmaceutically acceptable carrier comprises a lactose powder or a glucose powder.

46. The pharmaceutical composition of claim 44 or 45, wherein the TNFα antibody, or antigen-binding portion thereof, is an antibody selected from the group consisting of a humanized antibody, a chimeric antibody, a human antibody, and a multivalent antibody.

47. The pharmaceutical composition of claim 44 or 45, wherein the TNFα antibody, or antigen-binding portion thereof, is selected from the group consisting of infliximab, golimumab, and adalimumab.

48. The pharmaceutical composition of claim 46, wherein the human anti-TNFα antibody, or antigen-binding portion thereof, dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

49. The pharmaceutical composition of claim 46, wherein the human TNFα antibody, or antigen-binding portion thereof, has the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

50. The pharmaceutical composition of claim 46, wherein the human TNFα antibody, or antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

51. The pharmaceutical composition of claim 46, wherein the human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

52. The pharmaceutical composition of any one of claims 48-51, comprising at least about 40 mg of the TNFα antibody, or antigen-binding portion thereof.

53. The pharmaceutical composition of any one of claims 48-51, comprising about 40-160 mg of the TNFα antibody, or antigen-binding portion thereof.

54. A dry powder inhaler (DPI) device for pulmonary administration of a TNFα inhibitor to a subject, the DPI device comprising
a reservoir comprising an inhalable powder or dry powder composition comprising the TNFα inhibitor, and
a means for introducing the inhalable powder or dry powder composition into the subject via inhalation.

55. The DPI device of claim 54, wherein the DPI device is either a single dose or a multidose inhaler.

56. The DPI device of claim 54, wherein the DPI device is either pre-metered or device-metered.

57. A metered dose inhaler (MDI) device for pulmonary administration of a TNFα inhibitor to a subject, the MDI device comprising
a pressurized canister comprising an aerosol comprising the TNFα inhibitor and a propellant, and
a means for introducing the aerosol into the subject via inhalation.

58. A container for use with a nebulizer device for pulmonary administration of a TNFα inhibitor to a subject, the container comprising a propellant-free inhalable solution or suspension comprising the TNFα inhibitor.

59. The device or container of any one of claims 54-58, wherein the TNFα inhibitor is a TNFα antibody, or antigen-binding portion thereof, or a fusion protein.

60. The device or container of claim 59, wherein the fusion protein is etanercept.

61. The device or container of claim 59, wherein the TNFα antibody, or antigen-binding portion thereof, is an antibody selected from the group consisting of a humanized antibody, a chimeric antibody, a human antibody, and a multivalent antibody.

62. The device or container of claim 61, wherein the TNFα antibody, or antigen-binding portion thereof, is selected from the group consisting of infliximab, golimumab, and adalimumab.

63. The device or container of claim 61, wherein the human TNFα antibody, or antigen-binding portion thereof, dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

64. The device or container of claim 61, wherein the human TNFα antibody, or antigen-binding portion thereof, has the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

65. The device or container of claim 61, wherein the human TNFα antibody, or antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

66. The device or container of claim 61, wherein the human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

67. The device or container of any one of claims 62-66, comprising at least about 40 mg of the TNFα antibody, or antigen-binding portion thereof.

68. The device or container of any one of claims 62-66, comprising about 40-160 mg of the TNFα antibody, or antigen-binding portion thereof.


SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  adalimumab light chain variable region CDR3
QRYNRAPYX

SEQ ID NO: 4 - protein, 12 aa, Artificial Sequence
  adalimumab heavy chain variable region CDR3
VSYLSTASSLDX

SEQ ID NO: 5 - protein, 7 aa, Artificial Sequence
  adalimumab light chain variable region CDR2
AASTLQS

SEQ ID NO: 6 - protein, 17 aa, Artificial Sequence
  adalimumab heavy chain variable region CDR2
AITWNSGHIDYADSVEG

SEQ ID NO: 7 - protein, 11 aa, Artificial Sequence
  adalimumab light chain variable region CDR1
RASQGIRNYLA

SEQ ID NO: 8 - protein, 5 aa, Artificial Sequence
  adalimumab heavy chain variable region CDR1
DYAMH

SEQ ID NO: 9 - protein, 107 aa, Artificial Sequence
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 light chain variable region CDR3
QKYNSAPYA

SEQ ID NO: 12 - protein, 9 aa, Artificial Sequence
  EP B12  light chain variable region CDR3
QKYNRAPYA

SEQ ID NO: 13 - protein, 9 aa, Artificial Sequence
  VL10E4 light chain variable region CDR3
QKYQRAPYT

SEQ ID NO: 14 - protein, 9 aa, Artificial Sequence
  VL100A9 light chain variable region CDR3
QKYSSAPYT

SEQ ID NO: 15 - protein, 9 aa, Artificial Sequence
  VLL100D2 light chain variable region CDR3
QKYNSAPYT

SEQ ID NO: 16 - protein, 9 aa, Artificial Sequence
  VLL0F4 light chain variable region CDR3
QKYNRAPYT

SEQ ID NO: 17 - protein, 9 aa, Artificial Sequence
  LOE5 light chain variable region CDR3
QKYNSAPYY

SEQ ID NO: 18 - protein, 9 aa, Artificial Sequence
  VLLOG7 light chain variable region CDR3
QKYNSAPYN

SEQ ID NO: 19 - protein, 9 aa, Artificial Sequence
  VLLOG9 light chain variable region CDR3
QKYTSAPYT

SEQ ID NO: 20 - protein, 9 aa, Artificial Sequence
  VLLOH1 light chain variable region CDR3
QKYNRAPYN

SEQ ID NO: 21 - protein, 9 aa, Artificial Sequence
  VLLOH10 light chain variable region CDR3
QKYNSAAYS

SEQ ID NO: 22 - protein, 9 aa, Artificial Sequence
  VL1B7 light chain variable region CDR3
QQYNSAPDT

SEQ ID NO: 23 - protein, 9 aa, Artificial Sequence
  VL1C1 light chain variable region CDR3
QKYNSDPYT

SEQ ID NO: 24 - protein, 9 aa, Artificial Sequence
  VL0.1F4 light chain variable region CDR3
QKYISAPYT

SEQ ID NO: 25 - protein, 9 aa, Artificial Sequence
  VL0.1H8 light chain variable region CDR3
QKYNRPPYT

SEQ ID NO: 26 - protein, 9 aa, Artificial Sequence
  LOE7.A light chain variable region CDR3
QRYNRAPYA

SEQ ID NO: 27 - protein, 12 aa, Artificial Sequence
  2SD4 heavy chain variable region CDR3
ASYLSTSSSLDN

SEQ ID NO: 28 - protein, 12 aa, Artificial Sequence
  VH1B11 heavy chain variable region CDR3
ASYLSTSSSLDK

SEQ ID NO: 29 - protein, 12 aa, Artificial Sequence
  VH1D8 heavy chain variable region CDR3
ASYLSTSSSLDY

SEQ ID NO: 30 - protein, 12 aa, Artificial Sequence
  VH1A11 heavy chain variable region CDR3
ASYLSTSSSLDD

SEQ ID NO: 31 - protein, 12 aa, Artificial Sequence
  VH1B12 heavy chain variable region CDR3
ASYLSTSFSLDY

SEQ ID NO: 32 - protein, 12 aa, Artificial Sequence
  VH1E4 heavy chain variable region CDR3
ASYLSTSSSLHY

SEQ ID NO: 33 - protein, 12 aa, Artificial Sequence
  VH1F6 heavy chain variable region CDR3
ASFLSTSSSLEY

SEQ ID NO: 34 - protein, 12 aa, Artificial Sequence
  3C-H2 heavy chain variable region CDR3
ASYLSTASSLEY

SEQ ID NO: 35 - protein, 12 aa, Artificial Sequence
  VH1-D2.N heavy chain variable region CDR3
VSYLSTASSLDN

SEQ ID NO: 36 - DNA, 321 bp, Artificial Sequence
  adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt